SEQUENCE LISTING (16 sequences)

SEQ ID NO: 1 - DNA, 1888 bp, Homo sapiens
  misc_feature (922)..(922): n is a, c, g, or t
aaagaagatgaaatagaaacttgtacttggcctcagaatgcctgtagaaaccttagcaattgaatccagcccttatgttataggctgagttaactgtggcccagaaagactatgtgatttgctcacagttcttgattcccagactggcactgcggtgatggtgtgtgatgaggtagtatcttagtaagaacacaatccagaagtcactgcctcgggggaatcccagctcagcttcttgctagcttgcgtaggctggttacttcacttcagctccctgaatctgctttcttatctctaaaataaaaataatagcttcttcctcagagtagttggtggaactgaaagaatacatgtaaagtgcttagtatgacacctgccacataatatgaactgaattattgtgaattatgataaatttgtcagatactggtttacaaatcggatgttagaataacatggaatcagtgtttcagtcattttactatacatatgcaatattttctacatttgatctcacttcagaaacaaaatactgccccccccattttacaaatgcatatttttttctcagcaataatgttcaagaacaagtgcttggcccatattttgttgtctttacatggctttctttaaataatggggatggatttattaaataacctcatgagtaattttcaaaatttccattaagatcttgattgaaattggatgaaaaatcatttctaagaaaaacccaatgaagtgtttttctttgccacatttgacaattgccttggacttggtaaagtaatcattactgtgttgagtacctccagtgccctccttgacgctgccttagaaaaggtagctgcttttgaatgacaggcaggaatttgttcgccttttaggttcagcctgtaggtgccctctgcaggaaatcagnaactagggttttggaagcagtcagggtggggttctcccttgtccctgcagcctcagcaaagactcaggcagtctggcaaaagcagtttcttcagcatacctaacagaacgcaagtttccatatgcctgatgcaaataatggcctccaaacgttaaaccttttttgagataaacttgttctttaattgccagcgcctgcagttaattttgattggctacactctggttaaaagaaaatgctttcgatgtgatatggcaaatttggagaaaagtaactcttttgctcccaaccagtcttccacaacttaaacttaatcgtcctgtcctttttctgctgccctcgtggagtgtaagtttttgagggagaccagcagaaactgactttccatatgcccctgaagaataacttctttgaatgcaaagaggtggggacacggaggatctgtcattacgggttattatgggtgggacccagagacgggagtgaagggagggtgtggcccgcgggtgggatctgtagagcagacaaaatatggggcccctggcgcttaaagttcagtttgtctctcttgagcttggagaaaatcatccgtagtgcctccccgggggacacgtagaggagagaaaagcgaccaagataaaagtggacagaagaataagcgagactttttatccatgaaacagtctcctgccctcgctccggaagagcgctgccgcagagccgggtccccaaagccggtaagatcgatgattcgctgtcctaccgcagccaggtgtgatggcctcttagtcccggtgaattcagggtcagcccctcccgcccccgtcctctcctccgcccgatgccacctccttccccagccgccggtgagctcccgctctgacagttccgcgctcagcgccctgcacccagttcccaggcgcacggccctggtcccgaccaccaccccggtcgcccggcgtccga

SEQ ID NO: 2 - DNA, 4165 bp, Homo sapiens
  misc_feature (3363)..(3363): n is a, c, g, or t
aagcttcatgagggcaggagtgcagttttgtttattactgtaacctcagagtagagaagcctggccacatagtagatgctagtattcgtttcctagggctgctgtaacagataccatacatgagttacttaaaacaacagaaatttattctttcacagtttaggtggccagaagtcccaaaccaagatataacaaattatatctgcaaagcctttatttccacagaaccacattctgaggttctgggtggacattaatttggcagggagtagtggggggacactaacctactacagtgctcagtaaatatttattgggtgaatgaaaattcagagtgtattctaagctgtaaaccccttggatgttttcatcacagtcttctcttaataactgctacagaacataaggattagtttgtggtcccagattttttaggctccaattctgcttctaccactttctacttgtgagctcttaggcaagctatttaactttgtaagcctcagtttcttctgtaaaatggtgacaataagaaataacagtaagtgcccaataagttttaactattattatgtgtctatagctattttaaagcactttctatatgtaatcctcataacaaccgtattaggtgagcctattacacttctcattttatgacaaaggaaactagatttgtttcaagaatgaaaaacctagaaaattatatgtcactttatgaagtgcctggcacatagtacatagaatagcttagctacaataatatagatatgtgactttacctcataactttaagacatctcaaattgattcacaaatcagaaaaaaaatctgaggcatcagatgtgaggtgaggtgaggtgaggcatctcagtttttgcacatatggttactgttattgagggagctccatgtcattgagggagctccacgtcattgagggaggactgggtgggatgctggccaaaggtaggggcatatttaggcagtgaaattgcagttatgggacccctctatagcactagatgtgtgagaggactaaaaataaaattcctttgaaatttcttacagggtcatatattacctccttcccaatgactactgtggtatattagagtaggggattgatgtcccagaaatattatgtacatcaaacagaagatctacacaaattgttttatcagatactgaatatatcaatgggctagagtgtattatagtacatatgggtatgttgttttcccccttttgactaaataaactctcccctctctgcaacaggtaaatttccagtttacctttttcttgctgtttttaattttttttattttgaaggctttgtatctttatatctcagctgaaaatattacattctaactccccaaagctattctcacattatttgcaagtattttttcatagtttacatgtttgtgtatttgtttaatacattccacaagactgaaagtcttggtgagggcaggaactctgtctaggttgttcatctggcagccagcaaacccaaaataagtattagttgaatgaatagctaaatagatgagccatggggtagcatatctgttttagctactgcttgcctgttatcatcctgggatgctgcttgcttctccgtgatctcttcttgttcttaaattgtcttcagttgtagtactaagtactaatttgatctgtaatgtgatatgtggctgaagtttgctctgtaaaatcaccgtttcatgaggtatattcaatatctttaatttttccataaatctcttcaaggtttgtgtgtgttttcttttaaattatctaactagttggatgtatgcttgaagtgctagacaataaaagtttcaataggctagaaatgtttctttttgtaaaattattttaagaaactagatgatggttgtcacttgaagctgaaatgcaaatgtagctagttttttttaaagaataattcaaataggtcattaaagattactatgagcacactgcatattcaaatcagtttgaatatgttttgagacttctaatagtttatgattcttgacattttaatgagcacactgcatattcaaatcagtttgaatatgttttgagacttctaatagtttatgattcttgacattttaaaagtttattataaagaatataaaacatctttaccctcattttttatgtcattaccttcatgcaaagaatacctcaggtattaattctggtgctgttggtaactagaactggttgggttttcttgctaaaaggaagtttaaaaaatgtttattttacaccattaatgcaattagctttagttctcagaggaactaaaagtggaaaagtgagggagactgatcgaccctatctcaaatccactgacgtagctactttaatttcatagtccctgtagcacctccaccagagggcagaaagtggaagagaaagaagatgaaatagaaacttgtacttggcctcagaatgcctgtagaaaccttagcaattgaatccagcccttatgttataggctgagttaactgtggcccagaaagactatgtgatttgctcacagttcttgattcccagactggcactgcggtgatggtgtgtgatgaggtagtatcttagtaagaacacaatccagaagtcactgcctcgggggaatcccagctcagcttcttgctagcttgcgtaggctggttacttcacttcagctccctgaatctgctttcttatctctaaaataaaaataatagcttcttcctcagagtagttggtggaactgaaagaatacatgtaaagtgcttagtatgacacctgccacataatatgaactgaattattgtgaattatgataaatttgtcagatactggtttacaaatcggatgttagaataacatggaatcagtgtttcagtcattttactatacatatgcaatattttctacatttgatctcacttcagaaacaaaatactgccccccccattttacaaatgcatatttttttctcagcaataatgttcaagaacaagtgcttggcccatattttgttgtctttacatggctttctttaaataatggggatggatttattaaataacctcatgagtaattttcaaaatttccattaagatcttgattgaaattggatgaaaaatcatttctaagaaaaacccaatgaagtgtttttctttgccacatttgacaattgccttggacttggtaaagtaatcattactgtgttgagtacctccagtgccctccttgacgctgccttagaaaaggtagctgcttttgaatgacaggcaggaatttgttcgccttttaggttcagcctgtaggtgccctctgcaggaaatcagnaactagggttttggaagcagtcagggtggggttctcccttgtccctgcagcctcagcaaagactcaggcagtctggcaaaagcagtttcttcagcatacctaacagaacgcaagtttccatatgcctgatgcaaataatggcctccaaacgttaaaccttttttgagataaacttgttctttaattgccagcgcctgcagttaattttgattggctacactctggttaaaagaaaatgctttcgatgtgatatggcaaatttggagaaaagtaactcttttgctcccaaccagtcttccacaacttaaacttaatcgtcctgtcctttttctgctgccctcgtggagtgtaagtttttgagggagaccagcagaaactgactttccatatgcccctgaagaataacttctttgaatgcaaagaggtggggacacggaggatctgtcattacgggttattatgggtgggacccagagacgggagtgaagggagggtgtggcccgcgggtgggatctgtagagcagacaaaatatggggcccctggcgcttaaagttcagtttgtctctcttgagcttggagaaaatcatccgtagtgcctccccgggggacacgtagaggagagaaaagcgaccaagataaaagtggacagaagaataagcgagactttttatccatgaaacagtctcctgccctcgctccggaagagcgctgccgcagagccgggtccccaaagccggtaagatcgatgattcgctgtcctaccgcagccaggtgtgatggcctcttagtcccggtgaattc

SEQ ID NO: 3 - DNA, 1817 bp, Mus musculus
aactctgaaatgaaagaaactcaactgagccttcaagatgggtgaacgtacacccactgttccatcacagactgcttaactctggccttcagggtggagttggcaaatgtgtctcctgggctctgggttctcagatggggttcctgtgggtggtggtgtgcgctgaggcagtgtctcagtcagaactcagtcacaaaatgccaccatctctggggagtgtcaggtcactgcctaccaaatgacctcagcgggttatgccagttgcctctcatctctaagttaaagagggtatgtgcaaaatgcttagtgcagagcccggcatgctggaacactcaggtttgtgaattgtgaaacccttgttagaaatggcatgcctcactgatgctggaataacttgaggtcaagtgtccctcgtctttaacgacacatagatgatattcccacactccatgtcttttcggaagcaaagtacccattaccacttccttccactcgtacaaatgtgcctttgtccacacaatgatactcaaggtcttattgtctgcttactatatgtagccttctctagtggagatgggctacagtataaccttgaataattattgaaattttgaaaattttgattaaaaatgcatttaaaactcacgtttaagaaaacttaaatttttgttatccttggcggttttttttttcaattttcgataaatatttaagaattgtcttgggcttcaatgcaacctttgctttggaatgtctacactgctttccttgacgctattagaatgcagctccctgtctgatgggcgcgaatttgctggcccctgatttcagcctgggcaggttcttgtcagacaatcgcaggcccgagttctcctagcccatgaagctcaaacagcaacgccgtttccccccctaaacttgtagcagaagacagatttgcaatttgtgtaacacgaataatgcctgccttcgaaacctgacatcttttcgaacacaacaaacttgtcattggatcgctagtgtctgaagacattcattttgatacttgcctggctagaatcctgtatgaggggatagactgttctagatgtgataatgagagttcggaagagagcagccctttacactgctcaacaaagttctgccctcaacaaagcagcaaaggccaaaagctgctgccccatagttgaagggttggtttgtttgttttttccctggtgtggaggatgggggcagtagactcttggctctccatctgccatagaaaaataacttttttgaaggctggggtgggtatggaggaactgtctcattaagggttatgggtaggacccagagacgtgagtgaagggagggagtggtctgcgggtggggtctgcccagcagacaaaatatggggctccaatcccctgagtataacttcactccgagtgaggagaaagacacccgtagtgcctctcctcgagatccatagagcagagaaaagcgaccgagataagagtggacagaggagaaagatatgttcacgaaacggccccccgcccttgctccggaggagtacagccgccgagctgacgccccaaagagggtaagatccaccctcctcgctcctccagcacacaggtcgcgtggcctgagtcctggtgacttcagagtcaaccttttttctccctgtcccttcctcctttcatcgggtgccacctccttcccgtccgtaggtcagtgagcacagacttctcagtggctcgttctagtccccaggcagacggtccctcactcctgtggcttggcgtcggagacgctggcagtcatgggca

SEQ ID NO: 4 - DNA, 391 bp, Homo sapiens
agtaactcttttgctcccaaccagtcttccacaacttaaacttaatcgtcctgtcctttttctgctgccctcgtggagtgtaagtttttgagggagaccagcagaaactgactttccatatgcccctgaagaataacttctttgaatgcaaagaggtggggacacggaggatctgtcattacgggttattatgggtgggacccagagacgggagtgaagggagggtgtggcccgcgggtgggatctgtagagcagacaaaatatggggcccctggcgcttaaagttcagtttgtctctcttgagcttggagaaaatcatccgtagtgcctccccgggggacacgtagaggagagaaaagcgaccaagataaaagtggacagaagaataagc

SEQ ID NO: 5 - DNA, 1283 bp, Homo sapiens
  misc_feature (623)..(623): n is a, c, g, or t
ataaaaataatagcttcttcctcagagtagttggtggaactgaaagaatacatgtaaagtgcttagtatgacacctgccacataatatgaactgaattattgtgaattatgataaatttgtcagatactggtttacaaatcggatgttagaataacatggaatcagtgtttcagtcattttactatacatatgcaatattttctacatttgatctcacttcagaaacaaaatactgccccccccattttacaaatgcatatttttttctcagcaataatgttcaagaacaagtgcttggcccatattttgttgtctttacatggctttctttaaataatggggatggatttattaaataacctcatgagtaattttcaaaatttccattaagatcttgattgaaattggatgaaaaatcatttctaagaaaaacccaatgaagtgtttttctttgccacatttgacaattgccttggacttggtaaagtaatcattactgtgttgagtacctccagtgccctccttgacgctgccttagaaaaggtagctgcttttgaatgacaggcaggaatttgttcgccttttaggttcagcctgtaggtgccctctgcaggaaatcagnaactagggttttggaagcagtcagggtggggttctcccttgtccctgcagcctcagcaaagactcaggcagtctggcaaaagcagtttcttcagcatacctaacagaacgcaagtttccatatgcctgatgcaaataatggcctccaaacgttaaaccttttttgagataaacttgttctttaattgccagcgcctgcagttaattttgattggctacactctggttaaaagaaaatgctttcgatgtgatatggcaaatttggagaaaagtaactcttttgctcccaaccagtcttccacaacttaaacttaatcgtcctgtcctttttctgctgccctcgtggagtgtaagtttttgagggagaccagcagaaactgactttccatatgcccctgaagaataacttctttgaatgcaaagaggtggggacacggaggatctgtcattacgggttattatgggtgggacccagagacgggagtgaagggagggtgtggcccgcgggtgggatctgtagagcagacaaaatatggggcccctggcgcttaaagttcagtttgtctctcttgagcttggagaaaatcatccgtagtgcctccccgggggacacgtagaggagagaaaagcgaccaagataaaagtggacagaagaataagc

SEQ ID NO: 6 - DNA, 4023 bp, Homo sapiens
  misc_feature (3363)..(3363): n is a, c, g, or t
aagcttcatgagggcaggagtgcagttttgtttattactgtaacctcagagtagagaagcctggccacatagtagatgctagtattcgtttcctagggctgctgtaacagataccatacatgagttacttaaaacaacagaaatttattctttcacagtttaggtggccagaagtcccaaaccaagatataacaaattatatctgcaaagcctttatttccacagaaccacattctgaggttctgggtggacattaatttggcagggagtagtggggggacactaacctactacagtgctcagtaaatatttattgggtgaatgaaaattcagagtgtattctaagctgtaaaccccttggatgttttcatcacagtcttctcttaataactgctacagaacataaggattagtttgtggtcccagattttttaggctccaattctgcttctaccactttctacttgtgagctcttaggcaagctatttaactttgtaagcctcagtttcttctgtaaaatggtgacaataagaaataacagtaagtgcccaataagttttaactattattatgtgtctatagctattttaaagcactttctatatgtaatcctcataacaaccgtattaggtgagcctattacacttctcattttatgacaaaggaaactagatttgtttcaagaatgaaaaacctagaaaattatatgtcactttatgaagtgcctggcacatagtacatagaatagcttagctacaataatatagatatgtgactttacctcataactttaagacatctcaaattgattcacaaatcagaaaaaaaatctgaggcatcagatgtgaggtgaggtgaggtgaggcatctcagtttttgcacatatggttactgttattgagggagctccatgtcattgagggagctccacgtcattgagggaggactgggtgggatgctggccaaaggtaggggcatatttaggcagtgaaattgcagttatgggacccctctatagcactagatgtgtgagaggactaaaaataaaattcctttgaaatttcttacagggtcatatattacctccttcccaatgactactgtggtatattagagtaggggattgatgtcccagaaatattatgtacatcaaacagaagatctacacaaattgttttatcagatactgaatatatcaatgggctagagtgtattatagtacatatgggtatgttgttttcccccttttgactaaataaactctcccctctctgcaacaggtaaatttccagtttacctttttcttgctgtttttaattttttttattttgaaggctttgtatctttatatctcagctgaaaatattacattctaactccccaaagctattctcacattatttgcaagtattttttcatagtttacatgtttgtgtatttgtttaatacattccacaagactgaaagtcttggtgagggcaggaactctgtctaggttgttcatctggcagccagcaaacccaaaataagtattagttgaatgaatagctaaatagatgagccatggggtagcatatctgttttagctactgcttgcctgttatcatcctgggatgctgcttgcttctccgtgatctcttcttgttcttaaattgtcttcagttgtagtactaagtactaatttgatctgtaatgtgatatgtggctgaagtttgctctgtaaaatcaccgtttcatgaggtatattcaatatctttaatttttccataaatctcttcaaggtttgtgtgtgttttcttttaaattatctaactagttggatgtatgcttgaagtgctagacaataaaagtttcaataggctagaaatgtttctttttgtaaaattattttaagaaactagatgatggttgtcacttgaagctgaaatgcaaatgtagctagttttttttaaagaataattcaaataggtcattaaagattactatgagcacactgcatattcaaatcagtttgaatatgttttgagacttctaatagtttatgattcttgacattttaatgagcacactgcatattcaaatcagtttgaatatgttttgagacttctaatagtttatgattcttgacattttaaaagtttattataaagaatataaaacatctttaccctcattttttatgtcattaccttcatgcaaagaatacctcaggtattaattctggtgctgttggtaactagaactggttgggttttcttgctaaaaggaagtttaaaaaatgtttattttacaccattaatgcaattagctttagttctcagaggaactaaaagtggaaaagtgagggagactgatcgaccctatctcaaatccactgacgtagctactttaatttcatagtccctgtagcacctccaccagagggcagaaagtggaagagaaagaagatgaaatagaaacttgtacttggcctcagaatgcctgtagaaaccttagcaattgaatccagcccttatgttataggctgagttaactgtggcccagaaagactatgtgatttgctcacagttcttgattcccagactggcactgcggtgatggtgtgtgatgaggtagtatcttagtaagaacacaatccagaagtcactgcctcgggggaatcccagctcagcttcttgctagcttgcgtaggctggttacttcacttcagctccctgaatctgctttcttatctctaaaataaaaataatagcttcttcctcagagtagttggtggaactgaaagaatacatgtaaagtgcttagtatgacacctgccacataatatgaactgaattattgtgaattatgataaatttgtcagatactggtttacaaatcggatgttagaataacatggaatcagtgtttcagtcattttactatacatatgcaatattttctacatttgatctcacttcagaaacaaaatactgccccccccattttacaaatgcatatttttttctcagcaataatgttcaagaacaagtgcttggcccatattttgttgtctttacatggctttctttaaataatggggatggatttattaaataacctcatgagtaattttcaaaatttccattaagatcttgattgaaattggatgaaaaatcatttctaagaaaaacccaatgaagtgtttttctttgccacatttgacaattgccttggacttggtaaagtaatcattactgtgttgagtacctccagtgccctccttgacgctgccttagaaaaggtagctgcttttgaatgacaggcaggaatttgttcgccttttaggttcagcctgtaggtgccctctgcaggaaatcagnaactagggttttggaagcagtcagggtggggttctcccttgtccctgcagcctcagcaaagactcaggcagtctggcaaaagcagtttcttcagcatacctaacagaacgcaagtttccatatgcctgatgcaaataatggcctccaaacgttaaaccttttttgagataaacttgttctttaattgccagcgcctgcagttaattttgattggctacactctggttaaaagaaaatgctttcgatgtgatatggcaaatttggagaaaagtaactcttttgctcccaaccagtcttccacaacttaaacttaatcgtcctgtcctttttctgctgccctcgtggagtgtaagtttttgagggagaccagcagaaactgactttccatatgcccctgaagaataacttctttgaatgcaaagaggtggggacacggaggatctgtcattacgggttattatgggtgggacccagagacgggagtgaagggagggtgtggcccgcgggtgggatctgtagagcagacaaaatatggggcccctggcgcttaaagttcagtttgtctctcttgagcttggagaaaatcatccgtagtgcctccccgggggacacgtagaggagagaaaagcgaccaagataaaagtggacagaagaataagc

SEQ ID NO: 7 - DNA, 20 bp, artificial sequence
  primer
aagcttcatgagggcaggag

SEQ ID NO: 8 - DNA, 26 bp, artificial sequence
  primer
gagctcgcttattcttctgtccactt

SEQ ID NO: 9 - DNA, 26 bp, artificial sequence
  primer
aagcttataaaaataatagcttcttc

SEQ ID NO: 10 - DNA, 26 bp, artificial sequence
  primer
aagcttagtaactcttttgctcccaa

SEQ ID NO: 11 - DNA, 42 bp, artificial sequence
  probe
cgtagaggagagaaaagcgaccaagataaaagtggacagaag

SEQ ID NO: 12 - DNA, 42 bp, artificial sequence
  probe
cgtagaggagagaaaagcgaccaacttaaaagtggacagaag

SEQ ID NO: 13 - DNA, 42 bp, artificial sequence
  probe
catagagcagagaaaagcgaccgagataagagtggacagagg

SEQ ID NO: 14 - DNA, 42 bp, artificial sequence
  probe
catagagcagagaaaagcgaccgacttaagagtggacagagg

SEQ ID NO: 15 - DNA, 27 bp, artificial sequence
  probe
cacttgataacagaaagtgataactct

SEQ ID NO: 16 - DNA, 27 bp, artificial sequence
  probe
cacttcttaacagaaagtcttaactct

(19) United States
(12) Patent Application Publication
(10) Pub. No.: US 2003/0223976 A1
(43) Pub. Date: Dec. 4, 2003

(54) Control sequences of the human corin gene

(75) Inventors: Junliang Pan, El Cerrito, CA (US); Qingyu Wu, Lafayette, CA (US)

Correspondence Address: BERLEX BIOSCIENCES, PATENT DEPARTMENT, 2600 HILLTOP DRIVE, P.O. BOX 4099, RICHMOND, CA, 94804-0099, US

(73) Assignee: Schering Aktiengesellchaft, Berlin (DE)

(21) Appl. No.: 10/447,476

(22) Filed: May 28, 2003

Related U.S. Application Data
(60) Provisional application No. 60/384,108, filed on May 31, 2002.

(51) Int. Cl. A61K 48/00; C12Q 1/68; C07H 21/04

(52) U.S. Cl. 424/93.21; 514/44; 435/6; 536/23.2

(57) ABSTRACT

This invention provides a novel expression control region isolated from mammalian corin genes. This control region preferentially activates transcription in cardiac cells. Methods and compositions are provided to employ this control region for identification of agents capable of modulating corin expression and for treatment of cardiac disease.

This application claims the benefit of U.S. Provisional Application Serial No. 60/384,108, filed May 31, 2002, which is incorporated herein in full by reference.

### FIELD OF THE INVENTION

This invention provides a novel expression control region isolated from mammalian corin genes. This control region preferentially activates transcription in cardiac cells. Methods and compositions are provided to employ this control region for identification of agents capable of modulating corin expression and for treatment of cardiac diseases.

### BACKGROUND

Corin, a cardiac transmembrane serine protease, plays an important role in the conversion of pro-atrial natriuretic peptides (pro-ANP) to ANP (Yan, W. et al. (2000) PNAS, 97: 8525-8529; Wu et al. (2002) J. Biol. Chem. 277:16900-16905). ANP is a cardiac hormone that reduces high blood pressure by promoting salt excretion, increasing urinary output, decreasing blood volume, and relaxing vessel tension in a receptor dependent manner. ANP has been implicated in major cardiovascular diseases such as hypertension and cardiac failure (Burnett, J. C. et al. (1986) Science, 231:1145-1147). In knockout mice, deficiency in either ANP or its receptor causes spontaneous hypertension (John, S. W. et al. (1995) Science 267:679-681; John, S. W. et al. (1996) Am. J. Physiol. 271, R109-R114; Lopez et al. (1995) Nature 378:65-68). It is recognized that the activation step of converting pro-ANP to ANP is critical in the regulation of the cardiac hormone.

Corin has a predicted structure of a type 11 transmembrane protein containing two frizzled-like cysteine rich motifs, eight LDL receptor repeats, a macrophage scavenger receptor-like domain, and a trypsin-like protease domain in the extracellular region (Yan et al. (1999) J. Biol. Chem. 274:14926-14935). The overall topology of corin is similar to that of other type 11 transmembrane serine proteases including hepsin, enterokinase, MT-SP1/matriptase, human airway trypsin-like protease, TMPRSS2, TMPRSS3/TADG-12, TMPRSS4, MSPL, and Stubble-stubbloid. The similar topologies as well as distinct modular structures suggest that these proteins comprise a gene family evolved by duplication and rearrangement of ancestral exons.

The human gene spans >200 kb and contains 22 exons. The intron/exon boundaries are well conserved among species with most exons encoding structural domains. Cloning of both mouse and human cDNA encoding the corin protein has been previously reported (Yan et al. ibid). Northern analysis showed that corin mRNA is highly expressed in the human heart. By fluorescence in situ hybridization analysis, the human corin gene was mapped to the short arm of chromosome 4 (4p12-13) where a congenital heart disease locus, total anomalous pulmonary venous return had been previously localized.

### SUMMARY OF THE INVENTION

The present invention is related to the isolation, cloning and identification of the expression control regions of the mammalian corin gene, including the promoter and other regulatory elements, and the use of this cardiac-specific expression control region to identify novel agents that modulate corin gene expression and to treat heart disease.

Toward these ends, it is an object of the present invention to provide an isolated polynucleotide comprising a corin expression control region, wherein the control region modulates transcription of any heterologous polynucleotide to which it is operably linked, including, but not limited to, the human corin gene.

The corin expression control region directs cardiac-specific transcription of the heterologous polynucleotides to which it is operably linked, comprises one or more transcription regulation elements, selected from the group consisting of GATA, Tbx-5, NKx2.5, Krüppel-like transcription factor, or NF-AT binding sites, and is capable of binding transcription proteins, e.g. GATA-4.

It is a further object of the invention to provide polynucleotides comprising a human corin expression control region. A preferred polynucleotide of the invention is located at nucleotides −4037 to −15 (SEQ ID NO: 6), more preferred at nucleotides −1297 to −15 (SEQ ID NO: 5), and still more preferred at nucleotides −405 to −15 (SEQ ID NO: 4), where the numbering is relative to the translation initiation site (ATG) of the human corin gene or its complementary strand as shown in FIG. 8 (SEQ ID NO: 2).

In accordance with this aspect of the invention there are also provided fragments and variants of these polynucleotides.

It is another object of the invention to provide vectors comprising the corin expression control region, or fragments or variants thereof. In further embodiments, the vector also comprises a hererologous polynucleotide, e.g. corin, operably linked to the corin expression control region. In accordance with this aspect of the invention, there are also provided host cells transfected with such vectors, and methods of expressing products encoded by such heterologous polynucleotides.

It is another object of the invention to provide pharmaceutical compositions comprising a vector containing the corin expression control region operably linked to a heterologous polynucleotide or a host cell transfected with such a vector, in a pharmaceutically acceptable carrier.

It is another object of the invention to provide a method of identifying an agent which can modulate the expression of a human corin gene in a cell, wherein the method comprises:

(a) producing a recombinant vector in which an isolated polynucleotide comprising a mammalian corin expression control region is operably linked to a reporter gene;

(b) transfecting the cell with the recombinant vector;

(c) treating the cell with the agent;

(d) measuring the level of transcription of the reporter sequence in the treated cell; and

(e) comparing the level of expression of the reporter sequence in the presence of the agent to the level of expression in a transfected control cell which has not been treated with the agent.

In a preferred embodiment, cardiac myocyte cells are used.

It is another object of the invention to provide a method for modulating the cardiac-specific expression of a gene in a human subject, the method comprising:

(a) producing a recombinant vector in which an isolated polynucleotide comprising a mammalian corin expression control region is operably linked to a heterologous polynucleotide; and

(b) administering the vector in a therapeutically effective amount to the subject.

A preferred embodiment of this aspect of the invention is a vector in which the heterologous polynucleotide encodes corin. Also preferred are embodiments in which the corin expression control region is selected from the polynucleotides having the sequences of SEQ ID NO: 4, SEQ ID NO: 5 or SEQ ID NO: 6.

It is another object of the invention to provide a method for treating congestive heart failure, hypertension or myocardial infarction in a human subject, the method comprising administering a therapeutically effective amount of an isolated polynucleotide comprising a corin expression control region, operably linked to a gene selected from the group consisting of corin, atrial natiuretic peptide (ANP), B-type natriuretic peptide, phosphonolamban, angiotensin converting enzyme (ACE), or dominant negative forms of these genes, to the subject. Alternatively, the corin expression control region may be operably linked to a polynucleotide which encodes an antisense RNA molecule.

In a preferred embodiment of this aspect of the invention the gene selected is corin.

It is another aspect of the invention to provide a method of treating a human subject with heart failure, the method comprising:

(a) producing a recombinant vector in which an isolated polynucleotide comprising a mammalian corin expression control region, is operably linked to a polynucleotide encoding a polypeptide selected from the group consisting of ANP, B-type natriuretic peptide, phosphonolamban, ACE, or dominant negative forms of these genes; and

(b) administering the recombinant vector, in a pharmaceutically acceptable carrier, to the subject.

### DESCRIPTION OF THE FIGURES

FIG. 1. Organization of the mammalian corin genes. Organization of the (A) human and (B) mouse corin genes is shown. Two BAC clones were sequenced by a shot-gun strategy and the sizes of their assembled insert sequences are indicated. Vertical bars indicate exons. A plasmid clone used for subcloning from BAC 26540 in the human gene is indicated by restriction enzyme sites (H, Hind III; E, EcoRI). The insert of the subclone was sequenced by a primer extension method using automated sequencing. At the bottom are depicted the positions of the BAC clones, Contigs, and a plasmid clone that span the corin gene.

FIG. 2. Intron-exon boundary positions relative to the protein domain of corin. Exons 1 through 22 (upper panel) of the corin gene are aligned with their corresponding protein domains. TM, transmembrane domain; Frizzled, the frizzled-like cysteine rich domain; LDLR, LDL receptor repeats; SRCR, scavenger receptor cysteine-rich domain; H, D, and S, the His, Asp, and Ser residues of the catalytic triad of the protease domain.

FIG. 3. Alignment of the 5′-flanking regions of the human (SEQ ID NO: 1) and mouse (SEQ ID NO: 3) corin genes. The 5′-flanking region, exon 1 and part of intron 1 are aligned between the human and murine genes. The numbering is relative to the translation initiator ATG (bold-type and italics). The numbers indicated are different between human and mouse, because of the divergence in the first exons. An arrowhead indicates the junction between the first exon and intron of the human corin gene, and the donor splice sequence of human intron 1 is underlined. The putative regulatory sequences are indicated and bold-typed (Tbx5 site for binding to Tbx5, a T-box containing transcription factor; NF-AT, a binding site for nuclear factor of activated T cells; GATA, a binding element for GATA proteins; GT box for binding to the Krüppel-like factors; TATA box for binding to basal transcription factor TFIID; and NKE, a binding motif for Nxk2.5). The NKE sequence, which overlaps with the proximal GATA sequence, is underlined.

FIG. 4. Functional analysis of corin gene promoter activity in cultured cardiomyocytes. Reporter constructs containing serially truncated segments of the 5′-flanking region of human and murine corin genes linked to the luciferase gene are diagramed (Panel A). The locations of putative regulatory elements are indicated. These constructs were co-transfected into mouse HL-5 cells with pRL-SV40, a Rellina luciferase-expressing plasmid driven by the SV40 viral promoter. The luciferase activity expressed by each construct was normalized to the activity of Rellina luciferase expressed by pRL-SV40 for each transfection. Each transfection experiment was performed in triplicate for each construct. The data represent the means±S.D. of three independent experiments (Panel B).

FIG. 5. Cardiac-specific expression of the 5′-flanking sequences from the human and murine corin genes. Cardiomyocytes HL5 cells and epitheloid HeLa cells were transfected with the indicated constructs, each along with the control construct pRL-SV40. Luciferase and Rellina activities are expressed as light units per 20 uL-aliquot of the cell extracts from the transfected cells. Each transfection experiment was performed in triplicate. The data represent the means±S.D. of three independent experiments.

FIG. 6. Binding of nuclear proteins to the regulatory sequence encompassing the proximal GATA element.

A: Sequences of the upper strand oilgonucleotides used as probes and competitors. The GATA motifs in each sequence (SEQ ID NOS: 11 and 13, for human and mouse, respectively) are in bold, and the mutated nucleotides (SEQ ID NOS: 12 and 14, for human and mouse, respectively) are in italics. The human and murine proximal GATA elements are from the indicated regions of the corin 5′-flanking sequences. The consensus GATA probe (SEQ ID NO: 15) containing two GATA motifs is derived from human T-cell receptor specific enhancer region.

B: The labeled consensus GATA probe (SEQ ID NO: 15) or its mutant probe (SEQ ID NO: 16) was incubated with nuclear extracts from HL-5 cells in the presence or absence of a 100-fold excess of the indicated unlabeled oligonucleotides. The arrow indicates a GATA-sequence dependent DNA-protein complex.

C: The labeled consensus GATA probe was incubated with nuclear extracts from HL-5 cells in the presence of antibodies against GATA proteins. The arrow indicates a DNA-protein complex whose formation was blocked by an antibody against GATA-4, but not by antibodies against GATA-1, -3,and -6.

D: The labeled human corin GATA element was incubated with nuclear extracts from HL-5 in the presence or absence of an antibody against GATA-4. The arrow indicates the DNA-protein complex whose formation was completely blocked in the presence of the anti-GATA-4 antibody.

FIG. 7. Mutational analysis of the proximal conserved GATA elements. The same mutations (GATA to CTTA) that abolish the binding of GATA-4 protein in EMSA were introduced into the luciferase reporter constructs driven by the 5′-flanking regions from −642 to −77 in mouse or from −405 to −15 in human. The mutant and wild type constructs were transfected into HL-5 cells, each along with the control construct pRL-SV40. The luciferase activity expressed by each construct was normalized into the Renilla luciferase activity expressed by pRL-SV40 for each transfection. The promoter activity of each mutant construct was expressed as a percentage of the corresponding wild-type construct. Each transfection experiment was performed in triplicate. The data represent the means±S.D. of three independent experiments.

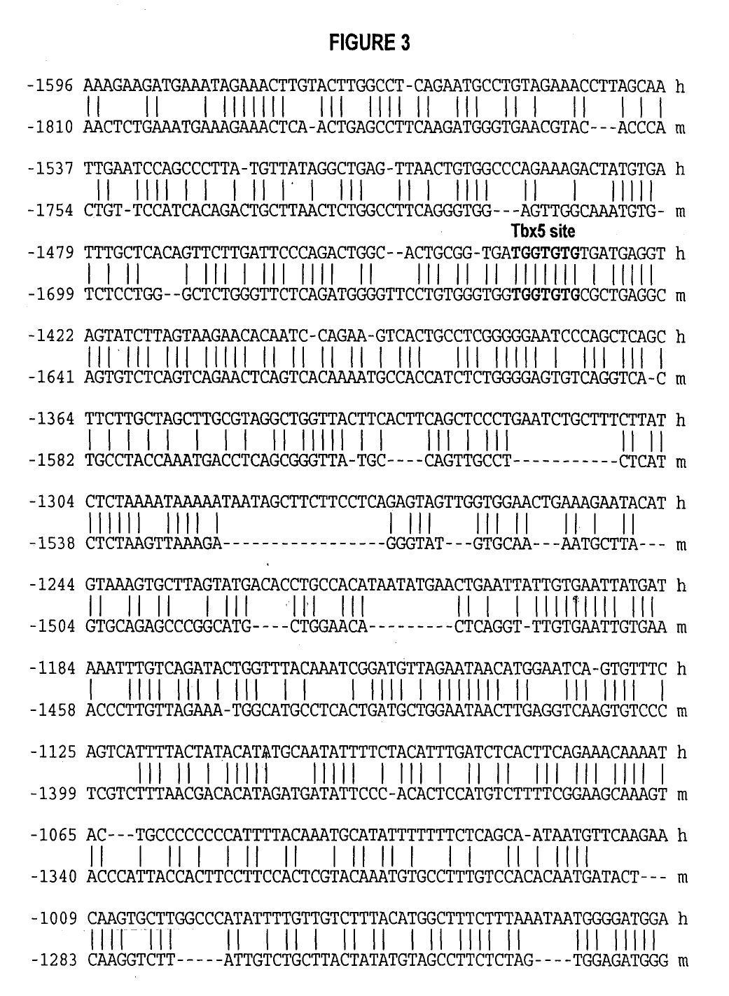

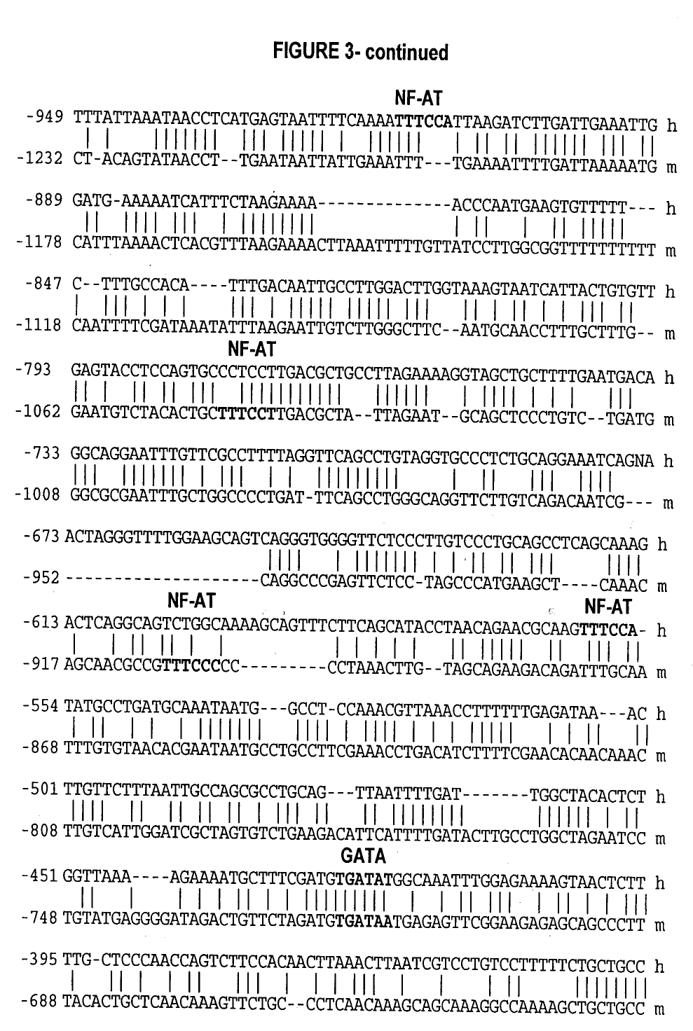

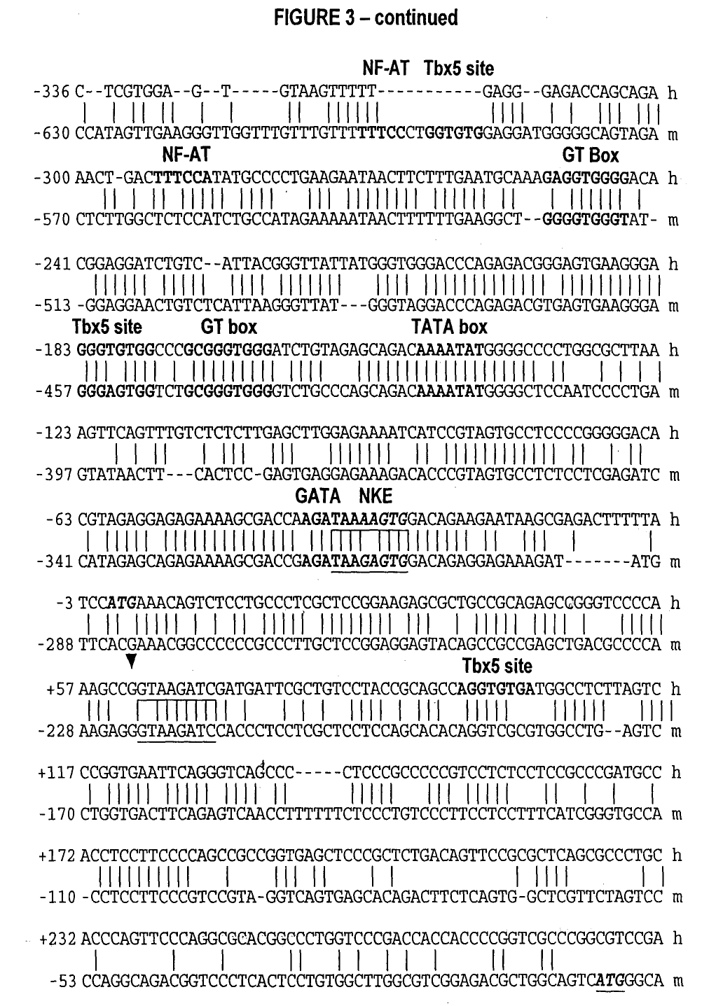

FIG. 8. Nucleotide sequence (SEQ ID NO: 2) of the 5′-flanking region of the human corin gene. The 4165-base pair sequence contains the 5′-flanking region, the first exon, and the beginning of intron 1 (in lower case). All numbering is relative to the translational start site (ATG, in boldface and underlined). The putative regulatory elements are indicated in boldface. The abbreviations for the putative regulatory elements are described in the legend of FIG. 3.

### DETAILED DESCRIPTION OF THE INVENTION

The present invention is related to the isolation, cloning and identification of the expression control region of the cardiac-specific corin gene, including the promoter and other regulatory elements. The isolated corin expression control region, and fragments and variants thereof, have utility in constructing in vitro and in vivo experimental models for studying the modulation of corin gene expression and for identifying novel modulators of corin gene expression. The expression control region can also be used in gene therapy targeted to cardiac disease states, e.g. heart failure.

Definitions

As used in the specification, examples and appended claims, unless specified to the contrary, the following terms have the meaning indicated.

“Nucleic acid” or “polynucleotide” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucl. Acids Res. 19:5081; Ohtsuka et al. (1985) J. Biol. Chem. 260:2605-08; Rossolini et al. (1994) Mol. Cell. Probes 8:91-98). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

“Corin gene” refers to a gene encoding a contiguous amino acid sequence sharing about at least 60% (preferably 75%, 78%, 90%, and more preferably about 95%) identity with the human corin gene amino acid sequence as disclosed in Yan et al. (1999) J. Biol. Chem. 274: 14926-14935).

“Corin expression control region” or “expression control region” refers to a polynucleotide located within the upstream (5′) genomic sequence of the coding region of the naturally-occurring mammalian corin genes. In the human corin gene, the corin expression control region begins at nucleotide −4165 and ends at nucleotide −15 relative to the translation initiation site (ATG) of the corin gene or its complementary strand as shown in FIG. 8. The corin expression control region is capable of activating transcription of the corin gene in cardiac tissue (myocytes). The corin expression control region polynucleotides may range from 100 to 5000 nucleotides in length; particular embodiments of the functional human corin expression control region are 4023, 1283, or 391 nucleotides (SEQ ID NOS: 6, 5, and 4, respectively) in length. Corin expression control region polynucleotides are generally at least 70% homologous to these sequences. In some embodiments, corin expression control region polynucleotides are at least 75%, 80%, 85%, 90%, 92%, 95%, or 100% homologous to these sequences. The term “control region” does not include the initiation or termination codons and other sequences already described in Yan et al. ibid. The corin expression control region contains binding sites for a variety of transcriptional regulatory proteins, e.g. GATA-4, which can be linked in a way that is substantially the same as in nature or in an artificial way. The corin expression control region activates transcription of the corin gene or of other heterologous polynucleotides which are operably linked to it, particularly in a cardiac-specific manner.

“Cardiac-specific expression” means that a polynucleotide is transcribed at a greater rate in cardiac-derived cells than in non-cardiac cells. Thus, a corin expression control region will generally activate transcription of a linked polynucleotide at least 2-fold more efficiently in cardiac myocytes than in non-cardiac cells, where expression in each case is normalized to the transcription of another polynucleotide linked to the SV40 promoter/enhancer or other constitutive promoter.

“Variant(s)” of polynucleotides, as used herein, are polynucleotides that differ from the polynucleotide sequence of a reference polynucleotide. Generally, differences are limited so that the poluynucleotide sequences of the reference and the variant are closely similar overall and, in many regions, identical. The differences are such that the function of the polynucleotide is not altered, and if the polynucleotide normally encodes a polypeptide, the resultant polypeptide is either unchanged in amino acid sequence or, while possessing differences in amino acid sequence, is still functionally identical.

“Fragment(s)”, as used herein, refer to a polynucleotide having a polynucleotide sequence that entirely is the same as part, but not all, of the polynucleotide sequence of the aforementioned corin expression control region and variants thereof. Such fragments maintain the ability of the corin expression control region to direct cardiac-specific transcription of heterologous polynucleotides to which they are operably linked.

“Transcription initiation elements” refer to sequences in a promoter that specify the start site of RNA polymerase II. Transcription initiation elements may include TATA boxes, which direct initiation of transcription 25-35 bases downstream, or initiator elements, which are sequences located near the transcription start site itself. Eukaryotic promoters generally comprise transcription initiation elements and either promoter-proximal elements, distant enhancer elements, or both.

“Recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

“Enhancer” refers to a DNA regulatory region that enhances transcription. An enhancer is usually, but not always, located outside the proximal promoter region and may be located several kilobases or more from the transcription start site, even 3′ to the coding sequence or within the introns of the gene. Promoters and enhancers may alone or in combination confer tissue specific expression.

“Silencer” refers to a control region of DNA which when present in the natural context of the corin gene causes a suppression of the transcription from that promoter either from its own actions as a discreet DNA segment or through the actions of trans-acting factors binding to said elements and effecting a negative control on the expression of the gene. This element may play a role in the restricted cell type expression pattern seen for the corin gene, for example expression may be permissive in cardiomyocytes where the silencer may be inactive, but restricted in other cell types in which the silencer is active. This element may or may not work in isolation or in a heterologous promoter construct.

“Isolated” when referring, e.g., to a polynucleotide means that the material is removed from its original environment (e.g., the natural environment if it is naturally occurring), and isolated or separated from at least one other component with which it is naturally associated. For example, a naturally-occurring polynucleotide present in it natural living host is not isolated, but the same polynucleotide, separated from all of the coexisting materials in the natural system, is isolated. Such polynucleotides could be part of a composition, and still be isolated in that such composition is not part of its natural environment.

“Percent identity” or percent identical when referring to a sequence, means that a sequence is compared to a claimed element or described sequence after alignment of the sequence to be compared with the described or claimed sequence. The comparison of sequences and determination of percent identity and similarity between two sequences can be accomplished using a mathematical algorithim. A preferred non-limiting example of such a mathematical algorithim is described in Karlin et al. (1993) Proc. Natl. Acad. Sci. USA 90:5873-5877. Such an algorithim is incorporated into the NBLAST and XBLAST programs (version 2.0) as described in Altschul et al. (1997) Nucleic Acid Res. 25:3389-3402.

“High stringency” as used herein means, for example, incubating a blot overnight (e.g., at least 12 hours) with a long polynucleotide probe in hybridization solution containing, e.g., 5× SSC, 0.5% SDS, 100:g/ml denatured salmon sperm DNA and 50% formamide, at 42° C. Blots can be washed at high stringency conditions that allow, e.g., for less than 5% bp mismatch (e.g., wash twice in 0.1× SSC and 0.1% SDS for 30 min at 65° C.), thereby selecting sequences having e.g., 95% or greater sequence identity.

A polynucleotide is “expressed” when a DNA copy of the polynucleotide is transcribed into RNA.

A polynucleotide is “operably linked” to a corin expression control region when conjunction of the polynucleotide and the corin expression control region in a single molecule results in transcription of the polynucleotide, most preferably in cardiac-specific transcription. (in myocytes).

“Heterologous polynucleotide” refers to polynucleotides, other than a corin expression control region, which are operably linked to a corin expression control region and preferentially expressed in cardiac-specific cells. The linked polynucleotide encodes a therapeutically useful molecule, e.g. a polypeptide, an antisense RNA. The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid is separated from open reading frames that flank the gene and encode other proteins. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence that alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

See, e.g., Creighton, Proteins (1984).

An “expression vector” refers to a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to an expression control region, e.g. the corin expression control region.

“Pharmaceutically acceptable excipient” refers to an acceptable carrier, and any pharmaceutically acceptable auxiliary substance as required to be compatible with physiological conditions, which are non-toxic and do not adversely effect the biological activity of the pharmaceutical composition suspended or included within it. Suitable excipients would be compounds such as mannitol, succinate, glycine, or serum albumin.

“Therapeutically effective amount” refers to that amount of a compound of the invention, which, when administered to a subject in need thereof, is sufficient to effect treatment, as defined below, for patients suffering from, or likely to develop, cardiac diseases. The amount of a compound which constitutes a “therapeutically effective amount” will vary depending on the compound, but can be determined routinely by one of ordinary skill in the art having regard to his own knowledge and to this disclosure.

“Treating” or “treatment” as used herein covers the treatment of cardiac disease, and includes:

(a) preventing cardiac disease from occurring in a human, particularly when such human is predisposed to having these conditions;

(b) inhibiting cardiac disease, i.e. arresting its development; or

(c) relieving cardiac disease, i.e. causing regressing of the conditions.

### DETAILED DESCRIPTION OF THE INVENTION

The present invention is related to the cloning and identification of the expression control region of a mammalian corin gene (e.g. mouse, human), including the promoter and other regulatory elements and the use of this expression control region to identify agents that modulate corin gene expression and in the treatment of heart disease. In particular, the invention relates to polynucleotides which comprise a novel human corin expression control region and the ability of this control region to direct cardiac-specific expression of heterologous polynucleotides operably linked to it.

Isolation and Characterization of Corin Expression Control Region Polynucleotides

This invention relies on routine techniques in the field of recombinant genetics. Basic texts disclosing the general methods of use in this invention include Sambrook et al., Molecular Cloning, A Laboratory Manual (2^{nd} ed. 1989); Kriegler, Gene Transfer and Expression: A Laboratory Manual (1990); and Ausubel et al., Current Protocols in Molecular Biology (John Wiley and Sons, New York, N.Y., 1994).

Organization of the Mammalian Corin Gene

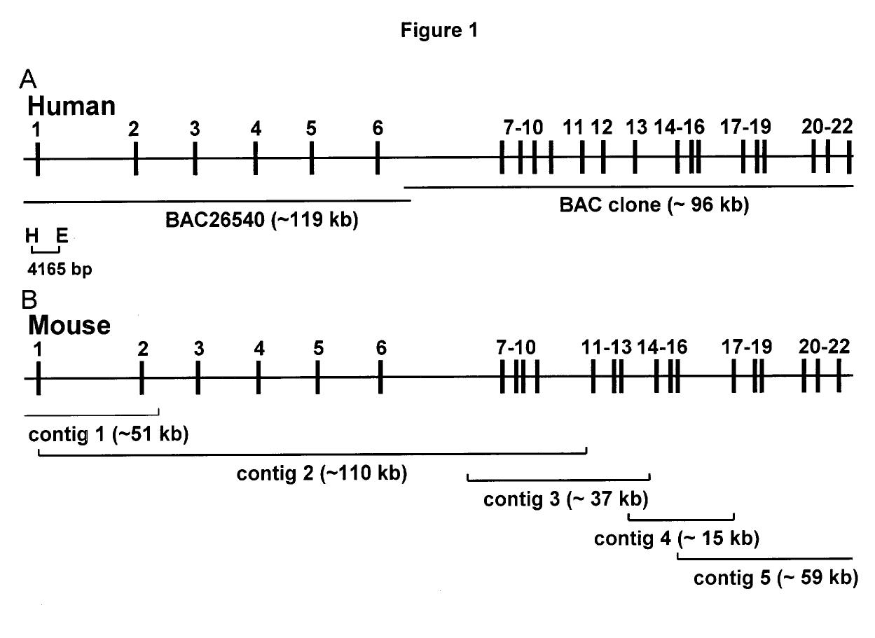

FIG. 1 depicts the organization of the human and murine corin genes and the locations of (bacterial artificial chromosome) BAC clones, contigs, and a plasmid clone containing the corin genes and their 5′-flanking regions. Both human and murine corin genes span at least 200 Kb and consist of 22 exons and 21 introns.

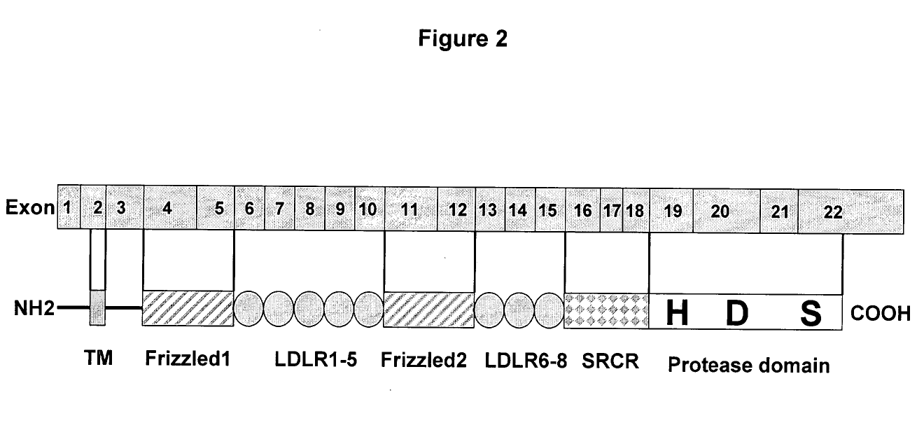

The corin cDNA sequence predicts a protein composed of a number of discrete domains. The boundaries between protein domains corresponds almost exactly to the exon/intron boundaries of the genomic structure, as illustrated schematically in FIG. 2. The cytoplasmic tail at the N-terminus is encoded by exon 1 and half of exon 2, followed by the transmembrane domain that is encoded by the other half of exon 2. The region between the transmembrane and the first Frizzled domain is encoded by exon 3. Each of the frizzled domains is encoded by two exons, each of the eight LDLRs by a single exon, and the scavenger receptor cysteine-rich domain by three exons. The protease domain at the C-terminus is encoded by exons 19 through 22, with the exon 19 coding for the sequence that includes the proteolytic activation site and the catalytic histidine residue. The exons 20 and 22 code for the sequences that include the other two catalytic residues aspartic acid and serine, respectively.

Cloning of the Corin Expression Control Region

To clone the human and murine corin genes and their 5′-flanking regions, specific oligonucleotides corresponding to the published corin cDNA sequences of these genes (Yan et. al. (1999) J. Biol. Chem. 274:14926-14935) were synthesized. These oligonucleotide primers were tested for amplifying specific products in PCR-based reactions using human or murine genomic DNA. The pairs of primers that successfully amplified specific PCR products were then used in a PCR-based screen to identify BAC clones containing the human or murine corin gene and/or their correspnding 5′-flanking regions. The identified positive BAC clones were either directly sequenced by a shotgun strategy or subcloned into pUC118 (PanVera/Takara, Madison, Wis.) for sequencing. The assembly of the shotgun sequences was done using the Staden package (Bonfield et al. (1995) Nucleic Acids Res. 23:4992-4999).

Four BAC clones, two each containing the human and murine corin genes, were obtained by PCR-based screening. Three BAC clones were sequenced by a shotgun strategy, and these sequences, in combination with available trace file information (http://www.ncbi.nim.nih.gov:80/Traces/trace.cqi, http://trace.ensembl.org), were used to assemble a contiguous sequences of 340 kb containing the human corin gene, and to determine sequences for 5 contigs for the murine corin gene. For the murine corin gene, the order of the 5 contigs was confirmed by the existence of several mated reading pairs in respective neighboring contigs. The distance of those allowed us to determine the gap size to be less than 500 bp, because the insert size of the public shotgun libraries was well defined. The structures of the human and murine corin genes were then analyzed. However, the 340-kb human genomic sequence did not contain the 5′-flanking region. An additional 4165 bp Hind III-EcoR I fragment was isolated from BAC 26540, which included the first 3919 bp of the 5′-flanking region, all of exon 1 and part of intron 1 (submitted to the GenBank™/EBI data Bank with the accession number AF521006).

The corin expression control region polynucleotides described herein were all derived from the 4165 bp Hind III-EcoR I fragment (see FIG. 8, SEQ ID NO: 2) isolated from BAC26540. These expression control region polynucleotides were obtained by a PCR-based method, or restriction enzyme digestion, or a combination of both. The 4023 bp corin expression control region polynucleotide (SEQ ID NO: 6) was amplified from the 4165 bp Hind III-EcoR I fragment or human genomic DNA using the primers F1 (5′-AAGCTTCATGAGGGCAGGAG-3′) (SEQ ID NO: 7) and R1 (5′-GAGCTCGCTTATTCTTCTGTCCACTT-3′) (SEQ ID NO: 8). Similarly, the 1283 bp corin expression control region polynucleotide (SEQ ID NO: 5) was amplified using the primers F2 (5′-AAGCTTATAAAAATAATAGCTTCTTC-3′) (SEQ ID NO: 9) and R1 and the 391 bp corin expression control region polynucleotide (SEQ ID NO: 4) was amplified using the primers F3 (5′-AAGCTTAGTAACTCTTTTGCTCCCAA-3′) (SEQ ID NO: 10) and R1.

Any mammalian tissue such as leukocytes, from which DNA may be easily extracted is a suitable source of genomic DNA for the isolation of mammalian corin expression control region polynucleotides.

Functional Corin Expression Control Region Polynucleotides

The corin expression control region polynucleotides described above are assayed for cardiac-specific transcriptional activity by operably linking a given expression control region polynucleotide to a reporter gene, transfecting the construct into cardiac myocytes, and assaying for the ability of the particular expression control region polynucleotide sequence to direct cardiac-specific transcription of the reporter gene. Reporter genes typically encode proteins with an easily assayed enzymatic activity that is naturally absent from the host cell. Typical reporter proteins for eukaryotic promoters include chloramphenicol acetyltransferase (CAT), firefly or Renilla luciferase, beta-galactosidase, beta-glucuronidase, alkaline phosphatase, and green fluorescent protein (GFP). A preferred reporter gene is firefly luciferase.

One system for assessing corin expression control region activity is transient or stable transfection into cultured cell lines. Assay vectors bearing corin expression control region polynucleotides operably linked to reporter genes can be transfected into any mammalian cell line for assays of promoter activity; for methods of cell culture, transfection, and reporter gene assay see Ausubel et al. (2000), supra; Transfection Guide, Promega Corporation, Madison, Wis. (1998). Corin expression control region polynucleotides may be assayed for cardiac-specific transcription activity by transfecting the assay vectors in parallel into cardiac-derived cell lines and non-cardiac derived cell lines. Typically, a control vector comprising a second reporter gene driven by a known promoter, e.g., Renilla luciferase driven by the SV40 early promoter/enhancer (pRL-SV40, Promega, Madison, Wis.) is co-transfected along with the assay vector to control for variations in transfection efficiency or reporter gene translation among the various cell lines.

Alternatively, corin expression control region polynucleotides driven transcription may also be detected by directly measuring the amount of RNA transcribed from the reporter gene. In these embodiments, the reporter gene may be any transcribable nucleic acid of known sequence that is not otherwise expressed by the host cell. RNA expressed from corin expression control region polynucleotide constructs may be analyzed by techniques known in the art, e.g., reverse transcription and amplification of mRNA, isolation of total RNA or poly A⁺ RNA, northern blotting, dot blotting, in situ hybridization, RNase protection, primer extension, high density polynucleotide array technology and the like.

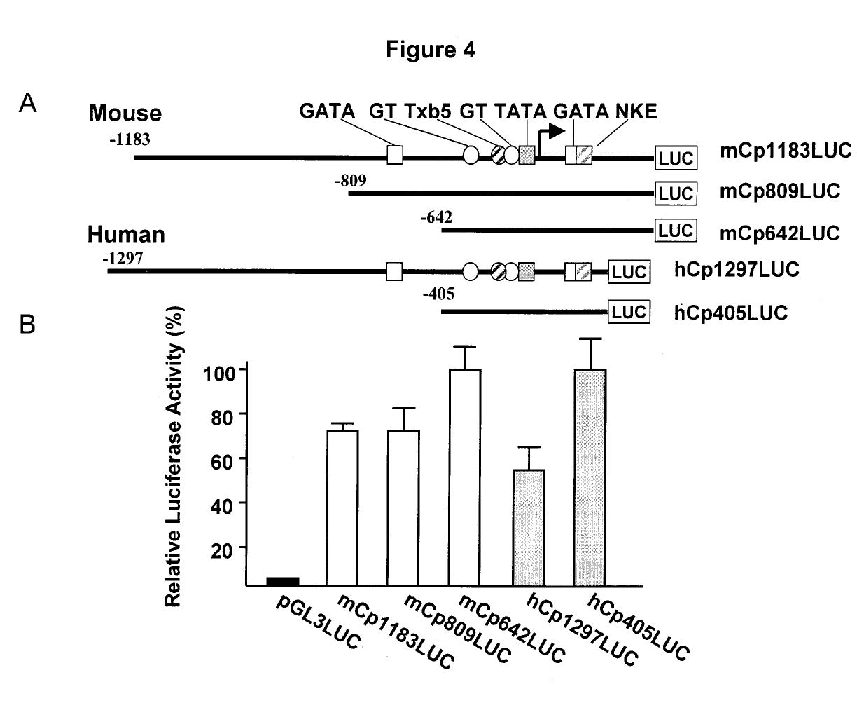

The ability of a corin expression control region polynucleotide sequence to activate transcription is typically assessed relative to a control construct. In one embodiment, the ability of a corin expression control region polynucleotide to activate transcription is assessed by comparing the expression of a reporter gene linked to a corin expression control region polynucleotide with the expression of the identical reporter gene not linked to such a sequence. Thus, in a preferred embodiment, the expression of luciferase is compared between pRL-SV40 and pRL-SV40 in which the corin expression control region polynucleotide sequences have been inserted 5′ of the luciferase gene (see Example 2, FIG. 4). In other embodiments, the activity of a corin expression control region polynucleotide may be compared with that of a known promoter. Thus, the activity of a reporter gene driven by a corin expression control region polynucleotide is compared to the activity of a reporter gene driven by a characterized promoter (e.g., the SV40 promoter/enhancer in pGL3-Control, Promega, Madison, Wis.).

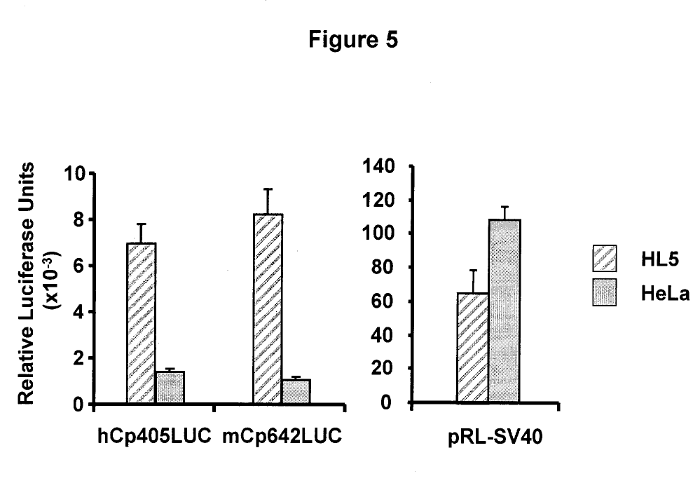

The cardiac-specificity of transcription directed by the corin expression control region is assessed by comparing the transcription of a reporter gene in cardiac-derived and non-cardiac derived cells. Suitable cardiac-derived cell lines for assessing cardiac-specific transcription are AT-1 (Claycomb et al. (1998) Proc. Natl. Acad. Sci. 95:2979-2984), HL-1 (Lanson et al.(1992) Circulation 85:1835-1841), and HL-5 (Wu et al. (2002) J. Biol. Chem. 277:16900-16905). A preferred cell line is the HL-5 cell line. Any readily transfectable mammalian cell line may be used to assay corin expression control region activity in non-cardiac cells (e.g., HeLa cells, ATCC No. CCL2). In Example 3 (FIG. 5), the cardiac-specific activity of both human (hCp405LUC) and mouse (mCp642LUC) corin expression control region polynucleotides is demonstrated by comparing firefly luciferase expression from vectors with and without these expression control region fragments in HL-5 and HeLa cell lines. For each assay, corin expression control region activity is normalized to co-transfected SV40 promoter activity (i.e., pGL3-Contol) to control for variability between the cell lines.

Once cardiac-specific transcriptional activity has been demonstrated in a corin expression control region polynucleotide, deletions, mutations, rearrangements, and other sequence modifications may be constructed and assayed for cardiac-specific transcription in the assays of the invention. Such derivatives of corin expression control region polynucleotides are useful to generate more compact promoters, to decrease background expression in non-cardiac cells, to eliminate repressive sequences, or to identify novel cardiac-specific transcriptional regulatory proteins. The human and rodent corin expression control region sequences may be compared to identify conserved transcription regulatory elements, including those that confer cardiac-specific expression.

Corin expression control region sub-fragments and derivatives may be constructed by conventional recombinant DNA methods known in the art. One such method is to generate a series of deletion derivatives within the corin expression control region sequence (Example 2). By comparing the transcriptional activity of a deletion series, the elements that contribute to or detract from cardiac-specific transcription may be localized. Based on such analyses, improved derivatives of corin expression control region polynucleotides may be designed. For example, corin expression control region elements may be combined with cardiac-specific or ubiquitous regulatory elements from heterologous promoters to increase the cardiac specificity or activity of a corin expression control region polynucleotide.

Vectors and Host Cells

The present invention also relates to vectors which include polynucleotides of the present invention, host cells which are genetically engineered with vectors of the invention and the production of polypeptides of the invention by recombinant techniques. Such techniques are described in Sambrook et al., Molecular Cloning, A Laboratory Manual, Cold Spring Harbor Press, Plainview, N.Y., 1989 and Ausubel, F. M. et al., Current Protocols in Molecular Biology, John Wiley & Sons, New York, N.Y., 1989.

Host cells can be genetically engineered to incorporate polynucleotides which contain the corin expression control region of the present invention as well as polynucleotides which contain the corin expression control region operably linked to genes which encode corin or other polypeptides, so as to permit expression of the product encoded by the linked polynucleotide, e.g. corin. Polynucleotides may be introduced into host cells using well known techniques of infection, transduction, transfection, transvection and transformation. The polynucleotides may be introduced alone or with other polynucleotides. Such other polynucleotides may be introduced independently, co-introduced or introduced joined to the polynucleotides of the invention.

Thus, for instance, polynucleotides of the invention may be transfected into host cells with another, separate, polynucleotide encoding a selectable marker, using standard techniques for co-transfection and selection in, for instance, mammalian cells. In this case, the polynucleotides generally will be stably incorporated into the host cell genome.

Alternatively, the polynucleotides may be joined to a vector containing a selectable marker for propagation in a host. The vector construct may be introduced into host cells by the aforementioned techniques. Generally, a plasmid vector is introduced as DNA in a precipitate, such as a calcium phosphate precipitate, or in a complex with a charged lipid. Electroporation also may be used to introduce polynucleotides into a host. If the vector is a virus, it may be packaged in vitro or introduced into a packaging cell and the packaged virus may be transduced into cells. A wide variety of techniques suitable for making polynucleotides and for introducing polynucleotides into cells in accordance with this aspect of the invention are well known and routine to those of skill in the art. Such techniques are reviewed at length in Sambrook et al. cited above, which is illustrative of the many laboratory manuals that detail these techniques. In accordance with this aspect of the invention, the vector may be, for example, a plasmid vector, a single or double-stranded phage vector, a single or double-stranded RNA or DNA viral vector. Such vectors may be introduced into cells as polynucleotides, preferably DNA, by well known techniques for introducing DNA and RNA into cells. The vectors, in the case of phage and viral vectors, also may be and preferably are introduced into cells as packaged or encapsidated virus by well known techniques for infection and transduction. Viral vectors may be replication competent or replication defective. In the latter case viral propagation generally will occur only in complementing host cells.

Preferred among vectors, in certain respects, are those for expression of polynucleotides and polypeptides of the present invention. Generally, such vectors comprise cis-acting control regions effective for expression in a host operatively linked to the polynucleotide to be expressed. Appropriate trans-acting factors either are supplied by the host, supplied by a complementing vector or supplied by the vector itself upon introduction into the host.

The corin expression control region polynucleotides of the invention may be inserted into the vector by any of a variety of well-known and routine techniques. In general, a DNA sequence for expression is joined to an expression vector by cleaving the DNA sequence and the expression vector with one or more restriction endonucleases and then joining the restriction fragments together using T4 DNA ligase. Procedures for restriction and ligation that can be used to this end are well known and routine to those of skill. Suitable procedures in this regard, and for constructing expression vectors using alternative techniques, which also are well known and routine to those of skill, are set forth in great detail in Sambrook et al. cited elsewhere herein.

Uses of the Corin Expression Control Region

The corin expression control region polynucleotides of the present invention are useful for specifically expressing therapeutic molecules in cardiac-derived cells. Cardiac-specific expression of therapeutic molecules may be used, for example, to treat congestive heart failure, hypertension, and cardiac hypertrophy. Accordingly, vectors comprising therapeutic polynucleotides operably linked to the corin expression control region polynucleotides of the present invention can be constructed and administered to patients to treat cardiac diseases and to develop new and improved therapeutics.

Any therapeutic polynucleotide may be operably linked to a corin expression control region polynucleotide, including but not limited to, a polynucleotide encoding corin. Typically, a corin expression control region polynucleotide is included in an expression cassette and inserted 5′ of the therapeutic polynucleotide to be expressed. Corin expression control region polynucleotides may be positioned immediately proximal to the therapeutic polynucleotide, although corin expression control region polynucleotide enhancer elements may be positioned anywhere within several kilobases of the therapeutic polynucleotide, including at the 3′ end of the therapeutic polynucleotide and within introns. The ability of a corin expression control region polynucleotide to confer cardiac-specific transcription from a given position may be verified by positioning the corin expression control region polynucleotide in the appropriate configuration relative to a reporter gene, and assaying for cardiac-specific reporter gene activity as described herein.

The corin expression control region polynucleotide may be linked directly to the polynucleotide encoding a therapeutic molecule without additional sequences. In embodiments where the corin expression control region polynucleotide does not include the corin transcription initiation elements, additional elements such as a TATA box and transcription initiation sites should be provided. These may either be the transcription initiation elements native to the therapeutic gene, or derived from a heterologous eukaryotic or viral promoter. Additionally, the level of therapeutic gene expression may be increased by including enhancer and polyadenylation sequences from the therapeutic gene or from heterologous genes, so long as the cardiac-specificity of expression (as measured in the assays of the invention) is maintained.

Vectors for transfecting cardiac-derived cells in vitro and in vivo, methods of ensuring sustained expression in cardiac-derived cells in vivo, methods of operably linking therapeutic polynucleotides to cardiac-specific promoters, and methods of targeting vectors to cardiac cells in vitro or in vivo, administration routes, and dosages for treatment of cardiac disease with therapeutic vectors may be found in Tang et al. (2002) Methods 28:259-266; Phillips et al. (2002) Hypertension 39:651-655; Prentice et al. (1997) Cardiovas. Res. 35:567-574; Beggah A T et al. (2002) PNAS 99:7160-7165; Monte et al. (2003) J. Physiol. 546:49-61.

Accordingly, corin expression control region polynucleotides of the present invention can be used for cardiac-specific expression of a variety of therapeutic polynucleotides. Therapeutic polynucleotides expressed by corin expression control region polynucleotides are either active themselves (e.g., antisense and catalytic polynucleotides) or encode a protein which would have a therapeutic benefit.

Expression of antisense and catalytic ribonucleotides. One type of therapeutic polynucleotide that may be expressed by the corin expression control region polynucleotides is antisense RNA or iRNA (Fire, A. (1999) Trends. Genet 15:358-363; Sharp, P. (2001) Genes Dev. 15:485-490). In such embodiments, the corin expression control region polynucleotide is operably linked to a polynucleotide which, when transcribed by cellular RNA polymerases, is capable of binding to target mRNA. The derivation of an antisense sequence, based upon a cDNA sequence encoding a target protein is described in, for example, Stein & Cohen (1988) Cancer Res. 48:2659-68 and van der Krol et al. (1988) BioTechniques 6:958-76. The target protein will generally be a protein whose presence is thought to contribute to, or increase the chances of, cardiac disease, e.g. angiotension converting enzyme, angiotensin II receptor or NF-ATC (Stein et al. (1998) Amer. Heart J. 135:914-923; Levin et al. (1998) New Eng. J. Med. 339:321-328; Keating and Goa (2003) Drugs 63:47-70). Thus, cardiac-specific expression of the antisense molecule can preferentially reduce expression of these proteins in at-risk individuals. Successful use of cardiac-specific antisense expression has been described (Beggah A T et al. (2002) PNAS 99:7160-7165). Such an approach has proved successful in treating cardiac fibrosis and heart failure using cardiac-specific expression (Lee et al. (1966) Anticancer Res. 16:1805-11).

In addition to antisense polynucleotides, ribozymes can be designed to inhibit expression of target molecules. A ribozyme is an RNA molecule that catalytically cleaves other RNA molecules. Accordingly, corin expression control region polynucleotides of the present invention may be used to express ribozymes specifically in cardiac-derived cells by linking a polynucleotide encoding a ribozyme to a corin expression control region polynucleotide. Different kinds of ribozymes have been described, including group I ribozymes, hammerhead ribozymes, hairpin ribozymes, RNase P, and axhead ribozymes (see, e.g., Castanotto et al. (1994) Adv. in Pharmacology 25: 289-317 for a general review of the properties of different ribozymes). The general features of hairpin ribozymes are described, e.g., in Hampel et al. (1990) Nucl. Acids Res. 18:299-304; Hampel et al., European Patent Publication No. 0 360 257 (1990); U.S. Pat. No. 5,254,678. Methods of preparing ribozymes are well known to those of skill in the art (see, e.g., Wong-Staal et al., WO 94/26877; Ojwang et al.(1993) Proc. Natl. Acad. Sci. USA 90:6340-44; Yamada et al. (1994) Hum. Gene Ther. 1:39-45; Leavitt et al. (1995) Proc. Natl. Acad. Sci. USA 92:699-703; Leavitt et al. (1994) Hum. Gene Ther. 5: 1115-20; and Yamada et al. (1994) Virology 205:121-26).

Expression of therapeutic proteins: A wide variety of therapeutic proteins may be used to treat cardiac diseases. Accordingly, a corin expression control region polynucleotide of the present invention may be used to express polynucleotides encoding therapeutic proteins specifically in cardiac cells. Therapeutic proteins may be of prokaryotic, eukaryotic, viral, or synthetic origin. Where the therapeutic protein is not of mammalian origin, the coding sequence of the protein may be modified for maximal mammalian expression according to methods known in the art (e.g., mammalian codon usage and consensus translation initiation sites).

Therapeutic proteins may be operably linked to the corin expression control region polynucleotides to permit cardiac-specific expression and be successfully employed to treat cardiac diseases of any etiology, including (but not limited to) ischemic heart disease, hypertensive heart disease, valvular heart disease, myocarditis, Chagas cardiomyopathy and idiopathic cardiomyopathy. Such therapeutic proteins include, but are not limited to, proteins such as corin, which converts pro-atrial natiuretic peptide (pro-ANP) to ANP, ANP, which lowers blood volume and pressure by promoting sodium secretion and vasodilation, and B-type natriuretic peptide, (Stein et al. (1998) Amer. Heart J. 135:914-923; Levin et al. (1998) New Eng. J. Med. 339:321-328; Keating and Goa (2003) Drugs 63:47-70), as well as negative dominant forms of such genes, i.e. corin (Wu et al. (2002) J. Biol. Chem. 277:16900-16905).

Identification of Modulators of Corin Expression

The corin expression control region polynucleotides of the present invention can be used to identify novel modulators which are useful in the control of cardiac-related disease in mammals, especially in humans, examples being cardiovascular hypertension, congestive heart failure, or cardiomyopathy. Such modulators are useful in treating a host with abnormal levels of corin gene expression. The corin gene modulators may also be used to treat diseases and conditions affected by the level of corin gene expression, such as, but not limited to, mechanic stretch, blood volume, salt excretion, urinary output, and vasomotor tone. The modulators are also useful in mimicking human diseases or conditions in animals relating to the level of expression of selected polypeptides.

Specifically, agents that bind to and modulate such expression can be identified by their ability to cause a change in the transcriptional level of a reporter gene, e.g. luciferase, which has been operably linked to a corin expression control region polynucleotide, as previously described. (See Example 2).

Agents that are assayed in the above method can be randomly selected or rationally selected or designed. As used herein, an agent is said to be randomly selected when the agent is chosen randomly without considering any the specific sequences. An example of randomly selected agents is the use of a chemical library or a growth broth of an organism or plant extract (Bunin, et al. (1992) J. Am. Chem. Soc. 114:10997-10998 and referenced combined therein).

As used herein, an agent is said to be rationally selected or designed when the agent is chosen on a nonrandom basis that takes into account the sequence of the target site and/or its conformation in connection with the agent's action.

Gene Therapy

The present invention provides corin expression control region polynucleotides which can be transfected into cells for therapeutic purposes in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. Typically, the operable linkage of a corin expression control region polynucleotide and a second, therapeutically useful, polynucleotide elicits cardiac-specific expression of the second polynucleotide. The compositions are administered to a patient in an amount sufficient to elicit a therapeutic response in the patient. An amount adequate to accomplish this is defined as “therapeutically effective dose or amount.”

Such gene therapy procedures have been used to correct acquired and inherited genetic defects, cancers, and viral infection in a number of contexts. The ability to express therapeutically useful artificial genes in humans facilitates the prevention and/or cure of many important human diseases, including many diseases that are not amenable to treatment by other therapies (for a review of gene therapy procedures, see Anderson (1992) Science 256:808-13; Nabel & Felgner, TIBTECH (1993), Vol. 11, pp. 211-17; Mitani & Caskey, TIBTECH (1993), Vol. 11, pp. 162-66; Mulligan, Science (1993), Vol. 260, pp. 926-32; Dillon, TIBTECH (1993), Vol. 11, pp. 167-75; Miller, Nature (1992), Vol. 357, pp. 455-60; Van Brunt, Biotechnology (1998), Vol. 6, pp. 1149-54; Vigne, Restorative Neurol. Neurosci. (1995), Vol. 8, pp. 35-36; Kremer & Perricaudet, British Medical Bulletin (1995), Vol. 51, pp. 31-44; Haddada et al., in Current Topics in Microbiology and Immunology (Doerfler & Böhm eds., 1995); and Yu et al., Gene Therapy (1994), Vol. 1, pp. 13-26).

Delivery of the gene or genetic material into the cell is the first step in gene therapy-based disease treatment. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell.

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386, 4,946,787; and 4,897,355, and lipofection reagents are sold commercially (e.g., Transfectam™ and Lipofectin™). Cationic and neutral lipids that are suitable for efficient receptor-recognition lipofection of polynucleotides include those of Felgner, WO 91/17424 and WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995), Vol. 270, pp. 404-10; Blaese et al., Cancer Gene Ther. (1995), Vol. 2, pp. 291-97; Behr et al., Bioconjugate Chem. (1994), Vol. 5, pp. 382-89; Remy et al., Bioconjugate Chem. (1994), Vol. 5, pp. 647-54; Gao et al., Gene Therapy (1995), Vol. 2, pp. 710-22; Ahmad et al., Cancer Res. (1992), Vol. 52, pp. 4817-20; U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues. In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kbp of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. (1992), Vol. 66, pp. 2731-39; Johann et al., J. Virol. (1992), Vol. 66, pp. 1635-40; Sommerfelt et al., Virology (1990), Vol. 176, pp. 58-59; Wilson et al., J. Virol. (1989), Vol. 63, pp. 2374-78; Miller et al., J. Virol. (1991), Vol. 65, pp. 2220-24; PCT/US94/05700).

pLASN and MFG-S are examples are retroviral vectors that have been used in clinical trials (Dunbar et al., Blood (1995), Vol. 85, pp. 3048-57; Kohn et al., Nat. Med. (1995), Vol. 1, pp. 1017-23; Malech et al., Proc. Natl. Acad. Sci. USA (1997), Vol. 94, pp. 12133-38). PA317/pLASN was the first therapeutic vector used in a gene therapy trial (Blaese et al., Science (1995), Vol. 270, pp. 475-80). Transduction efficiencies of 50% or greater have been observed for MFG-S packaged vectors (Ellem et al., Immunol. Immunother. (1997), Vol. 44, pp. 10-20; Dranoff et al., Hum. Gene Ther. (1997), Vol. 1, pp. 111-23).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology (1987), Vol. 160, pp. 38-47; U.S. Pat. No. 4,797,368; WO 93/24641; Kotin, Hum. Gene Ther. (1994), Vol. 5, pp. 793-801; Muzyczka, J. Clin. Invest. (1994), Vol. 94, pp. 1351). Construction of recombinant AAV vectors are described in a number of publications, including U.S. Pat. No. 5,173,414; Tratschin et al., Mol. Cell. Biol. (1985), Vol. 5, pp. 3251-60; Tratschin et al, Mol. Cell. Biol. (1984), Vol. 4, pp. 2072-81; Hermonat & Muzyczka, Proc. Natl. Acad. Sci. USA. (1984), Vol. 81, pp. 6466-70; and Samulski et al., J. Virol. (1989), Vol. 63, pp. 3822-28.

Recombinant adeno-associated virus vectors (rAAV) are a promising alternative gene delivery system based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., Lancet (1998), Vol. 351, pp. 1702-03; Kearns et al., Gene Ther. (1996), Vol. 9, pp. 748-55).

Replication-deficient recombinant adenoviral vectors (Ad) are predominantly used in transient expression gene therapy, because they can be produced at high titer and they readily infect a number of different cell types. Most adenovirus vectors are engineered such that a transgene replaces the Ad E1a, E1b, and E3 genes; subsequently the replication defective vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. (1998), Vol. 9, pp. 1083-92). Additional examples of the use of adenovirus vectors for gene transfer in clinical trials include Rosenecker et al., Infection (1996), Vol. 241, pp. 5-10; Welsh et al., Hum. Gene Ther. (1995), Vol. 2, pp. 205-18; Alvarez et al., Hum. Gene Ther. (1997), Vol. 5, pp. 597-613; Topf et al., Gene Ther. (1998), Vol. 5, pp. 507-13; Sterman et al., Hum. Gene Ther. (1998), Vol. 9, pp. 1083-89.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA (1995), Vol. 92, pp. 9747-51, reported that Moloney murine leukemia virus can be modified to express human heregulin fused to gp70, and the recombinant virus infects certain human breast cancer cells expressing human epidermal growth factor receptor. This principle can be extended to other pairs of viruses expressing a ligand fusion protein and target cell expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Pharmaceutical Compositions and Administration

The present invention also relates to pharmaceutical compositions which may comprise the corin expression control region, or a vector comprising the expression control region, in combination with a pharmaceutically acceptable carrier. In one embodiment of the present invention, the pharmaceutically acceptable carrier is pharmaceutically inert.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed., 1994)) and the references cited therein for a discussion of how to isolate and culture cells from patients).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, or transdermal application.

Formulations suitable for oral administration can consist of: (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of compositions can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

Kits

The present invention further relates to pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

Transgenic Mice

The corin expression control region polynucleotides of the present invention may also be used to produce a transgenic mammal, preferably a mouse. Such a transgenic organism is useful, for example, for identifying and/or characterizing agents that modulate expression and/or activity of such a polynucleotide. Transgenic animals are also useful as models for cardiac disease states. The invention disclosed herein also relates to a non-human transgenic animal comprising within its genome one or more copies of the polynucleotides of the invention. The transgenic animals of the invention may contain within their genome multiple copies of the polynucleotides.

In a preferred embodiment, the transgenic animal comprises within its genome an expression control region of the human corin gene. A variety of non-human transgenic organisms are encompassed by the invention, including e.g., drosophila, C. elegans, zebrafish and yeast. The transgenic animal of the invention is preferably a mammal, e.g., a cow, goat, sheep, rabbit, non-human primate, or rat, most preferably a mouse.

Methods of producing transgenic animals are well within the skill of those in the art, and include, e.g., homologous recombination, mutagenesis (e.g., ENU, Rathkolb et al.(2000) Exp. Physiol., 85:635-644), and the tetracycline-regulated gene expression system (e.g., U.S. Pat. No. 6,242,667), and will not be described in detail herein. (See e.g., Wu et al, Methods in Gene Biotechnology, CRC 1997,pp. 339-366; Jacenko, O., Strategies in Generating Transgenic Animals, in Recombinant Gene Expression Protocols, Vol. 62 of Methods in Molecular Biology, Humana Press, 1997, pp 399-424]

The present invention also relates to a non-human knockout animal whose genome contains an expression control region of the human corin gene which is operationally linked to a reporter sequence and wherein said control region is effective to initiate, terminate, or regulate the transcription of the reporter sequence.

Functional disruption of the reporter sequence operatively linked with the control sequence can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the reporter sequence of an exogenous sequence into the reporter sequence which inactivates it. Examples of transgenic animals having functionally disrupted genes are well known, e.g., as described in U.S. Pat. Nos. 6,239,326, 6,225,525, 6,207,878.

Without further elaboration, it is believed that one skilled in the art can, using the preceding descriptions, utilize the present invention to its fullest extent. All examples were carries out using standard techniques, which are well known in the art, except where otherwise described in detail. Routine molecular biology techniques of the following examples can be carried out as described in standard laboratory manuals, such as Sambrook et al., Molecular Cloning: A Laboratory Manual, 2nd Ed.; Cold Spring Harbor Laboratory Press, Cold Spring Harbor, N.Y., 1989.

The following preferred specific embodiments are, therefore, to be construed as merely illustrative, and not limitative of the remainder of the disclosure in any way whatsoever. The entire disclosure of all applications, patents, and publications cited above are hereby incorporated by reference.

### EXAMPLE 1

### Isolation and Characterization of the Human and Mouse Corin Genes Including the 5′-Flanking Regions

To clone the human and murine corin genes and their 5′-flanking regions, specific oligonucleotides corresponding to the 5′- and 3′-ends of corin cDNA sequences were synthesized. These oligonucleotide primers were tested for amplifying specific products in PCR-based reactions using human or murine genomic DNA. PCR reactions were performed using PCR Reagent System (Life Technologies Inc.) with 30 cycles of amplification (1-min denaturation at 94° C., 1-min annealing at 50° C., and 1-min extension at 72° C.) and a final 7-min extension at 72° C. The pairs of primers that successfully amplified specific PCR products were then used in a PCR-based screen to identify BAC clones containing the human or murine corin gene and/or their expression control regions. DNA isolation from BAC clones was carried out according to the manufacturer's instruction (Incyte Genomics, Palo Alto, Calif.). The identified positive bacterial artificial chromosomes (BAC) clones were further confirmed by Southern analysis using ³²P-labeled human and murine corin cDNA probes. The BAC clones were either directly sequenced by a shotgun strategy or subcloned into pUC118 (PanVera/Takara, Madison, Wis.) for sequencing. The assembly of the shotgun sequences was performed using the Staden software package (MRC Laboratory of Molecular Biology; Bonfield et al. (1995) Nucleic Acid Res. 23:4992-4999).

Four BAC clones, two each containing the human and murine corin genes, were obtained by PCR-based screening. Three BAC clones were sequenced by a shotgun strategy using dye terminator chemistry. Combination of the shotgun data with the publicly available trace file information (http://www.ncbi.nlm.nih.gov:80/Traces/trace.cqi, http://trace.ensembl.org), contiguous sequences of 340 kb containing the human corin gene, and 5 contigs for the murine corin gene were assembled. The order of 5 contigs was confirmed by the existence of several mated reading pairs in respective neighboring contigs. The distance of those allowed us to determine the gap size to be less than 500 bp, because the insert size of the public shotgun libraries was well defined. The structures of the human and murine corin genes were then analyzed. The 340-kb human genomic sequence, however, did not contain the 5′-flanking region. An additional 4165 bp Hind III-EcoR I fragment was isolated from BAC 26540, which included the first 3919 bp of the 5′-flanking region, all of exon 1 and part of intron 1 (submitted to the GenBank™/EBI data Bank with the accession number AF521006).

The corin expression control region polynucleotides (SEQ ID NO: 4, SEQ ID NO: 5 and SEQ ID NO: 6) were all derived from the 4165 bp Hind III-EcoR I fragment (FIG. 8; SEQ ID NO: 2) isolated from BAC26540, using a PCR-based method, or restriction enzyme digestion, or a combination of both. For example, the 4023 bp corin expression control region polynucleotide (SEQ ID NO: 6) described herein was amplified from the 4165 bp Hind III-EcoR I fragment or human genomic DNA using the primers F1 (5′-AAGCTTCATGAGGGCAGGAG-3′) (SEQ ID NO: 7) and R1 (5′-GAGCTCGCTTATTCTTCTGTCCACTT-3′) (SEQ ID NO: 8). The 1283 bp corin expression control region polynucleotide (SEQ ID NO: 5) described herein was amplified from the 4165 bp Hind III-EcoR I fragment or human genomic DNA using the primers F2 (5′-AAGCTTATAAAAATAATAGCTTCTTC-3′) (SEQ ID NO: 9) and R1. The 391 bp corin expression control region polynucleotide (SEQ ID NO: 4) described herein was amplified from the 4165 bp Hind III-EcoR I fragment or human genomic DNA using the primers F3 (5′-AAGCTTAGTAACTCTTTTGCTCCCAA-3′) (SEQ ID NO: 10) and R1. Any mammalian tissue such as leukocytes from which DNA may be easily extracted is a suitable source of genomic DNA for the isolation of mammalian corin polynucleotides.

### EXAMPLE 2

### Promoter Activity of the 5′-Flanking Regions

The promoter activity of the 5′-flanking regions of the corin genes was examined by preparing reporter constructs in which serially truncated fragments of the 5′-flanking sequences of human or murine corin genes were linked to a promoterless luciferase gene (see FIG. 4A). The human corin promoter reporter constructs, hCp1297LUC (1283 bp corin expression control region (SEQ ID NO: ______) linked to the firefly luciferase gene) and hCp405LUC (391 bp corin expression control region (SEQ ID NO: ______) linked to the firefly luciferase gene), were generated in two steps: first, PCR-based cloning of the 5′-flanking region of human corin gene from −1297 or-405 to −15 (relative to the translation initiation codon ATG) using primers that bear restriction sites of Sac I and Hind III, respectively; and, second, insertion of the respective PCR products into the Sac I and Hind III sites of the pGL3-basic vector (Promega, Madison, Wis.).

Similarly, the murine corin promoter constructs, mCp1183LUC, mCp809LUC and mCp646LUC, were also made by the PCR-based cloning approach described above. Plasmids for these constructs were prepared using an EndoFree Plasmid Maxi kit (Qiagen, Valencia, Calif.). Transfection of HL-5 cells (Claycomb et al. (1998) Proc. Natl. Acad. Sci., USA 95:2979-2984) was carried out using a lipofectin-based method according to the manufacturer's instruction (Life Technologies). Briefly, 10 ug DNA of each of the corin reporter constructs plus 0.1 ug of pRL-SV40 (Promega, Madison, Wis.) was mixed with 20 ug of lipofectin in 1 ml of OPTI-MEM I reduced-serum medium. The mixture was incubated for 30 min at room temperature, and was then added to ˜70% confluent HL-5 cultured in one well of 6-well plates. After incubation for 6 h, the medium was replaced with fresh Ex-Cell 320 culture medium; and 30 h later, the transfected cells were harvested and assayed for firefly and Renilla luciferase activities. A dual luciferase activity assay was performed according to the manufacturer's instruction (Promega). Briefly, cell extracts were prepared by lysing the transfected cells with 250 ul of freshly diluted passive lysis buffer (Promega). The lysates were frozen and thawed once before centrifugation at 13,000 rpm for 5 min to pellet the cell debris. The supernatants were transferred to a fresh tube, and a 20-ul aliquot of the supernatants was assayed by a Dual-Luciferase Reporter Assay system. The luminescence of the samples was monitored by a Microplate Luminometer LB96 V (EG&G Berthold), which measured light production (relative light units) for a duration of 10 s. Each of the cell extracts was assayed in triplicate. Each transfection experiment for each construct was performed in triplicate. Firefly luciferase activity was normalized to the activity of Renilla luciferase.

As shown in FIG. 4B, human corin reporter constructs hCP1297LUC and hCP405LUC promoted luciferase activities that were significantly higher than background in pGL3-basic transfected cells. Similarly, murine receptor constructs mCp1183LUC, mCp809LUC, and mCp646LUC promoted significant luciferase activities comparable to those of the human constructs. These data suggest that the cis sequence responsible for most of the promoter activity is located between nucleotides −405 to −15 or nucleotides −646 to −77 in the human and murine corin genes, respectively.

### EXAMPLE 3

### Demonstration of Cardiac-Specific Expression

To determine whether the constructs mediate cardiac-specific expression, HeLa cells (ATCC No. CCL2), which do not express corin mRNA and protein were transfected with the constructs described above. In contrast to their high activities in HL-5 cells, constructs hCp405LUC and mCp646LUC had only minimal promoter activity in HeLa cells (FIG. 5). As a control, simultaneously transfected pRL-SV40 promoted higher levels of Renilla luciferase activity in HeLa than in HL-5 cells, indicating that HeLa cells were as readily transfected as HL-5 cells in these experiments. These results indicate that the 5′-flanking sequences from −405 to −15 of the human or −646 to −77 of the murine corin genes contain elements that are sufficient for specific expression in cultured cardiomyocytes.

### EXAMPLE 4

### Proximal GATA Elements that Bind to GATA-4 are Required for Optimal Function of the Corin Promoters

The 5′-flanking regions from nucleotides −405 to −15 in human or from nucleotides −646 to −77 in mouse were sufficient to promote high levels of gene expression in cultured cardiomyocytes but not in HeLa cells. This suggests that these regions contain regulatory elements responsible for the cardiomyocyte-specific expression. Inspection of these regions revealed a conserved GATA consensus sequence (designated as the proximal GATA sequences).

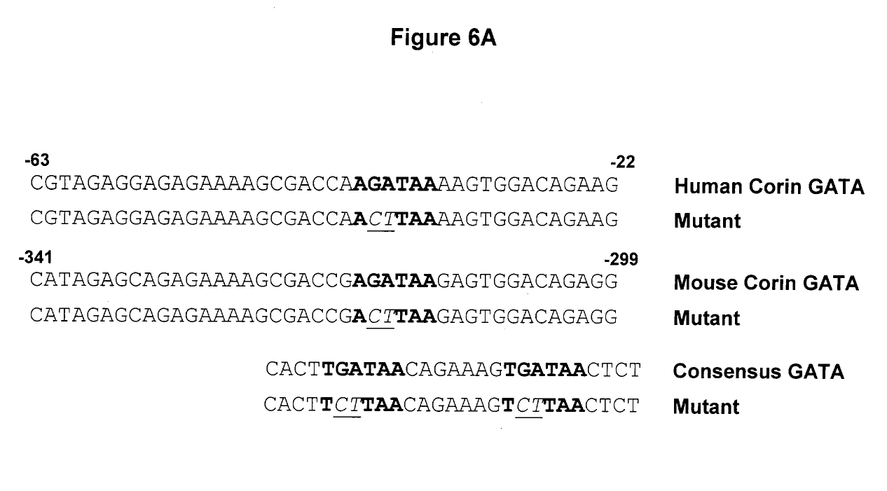

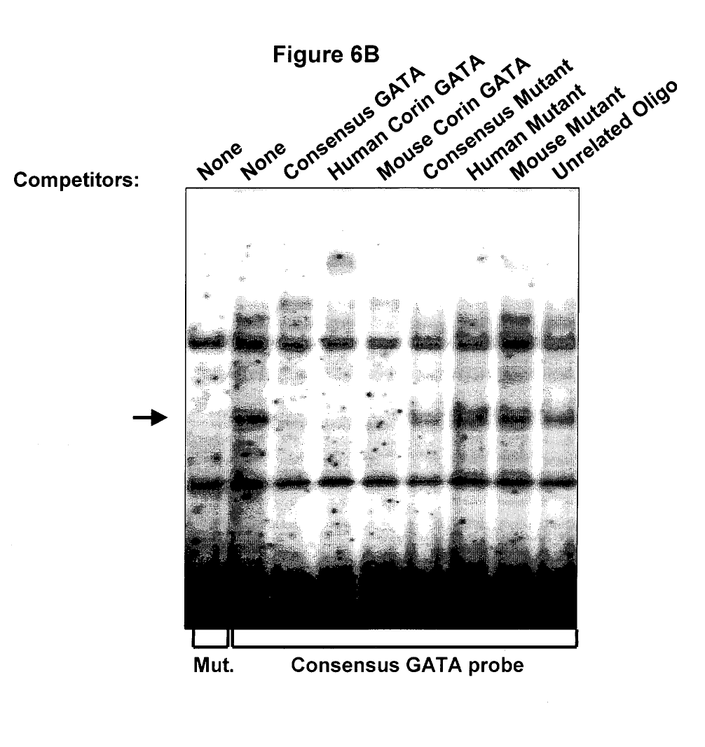

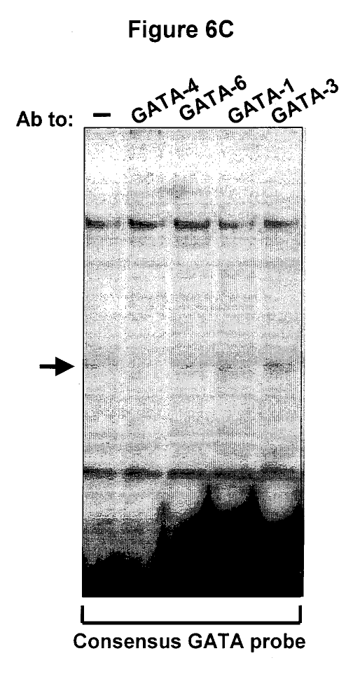

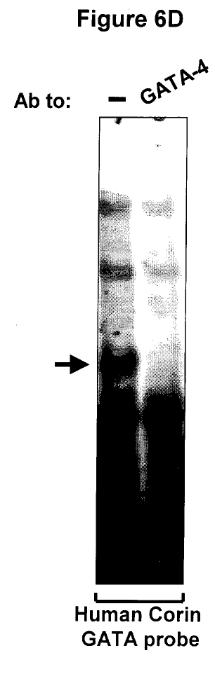

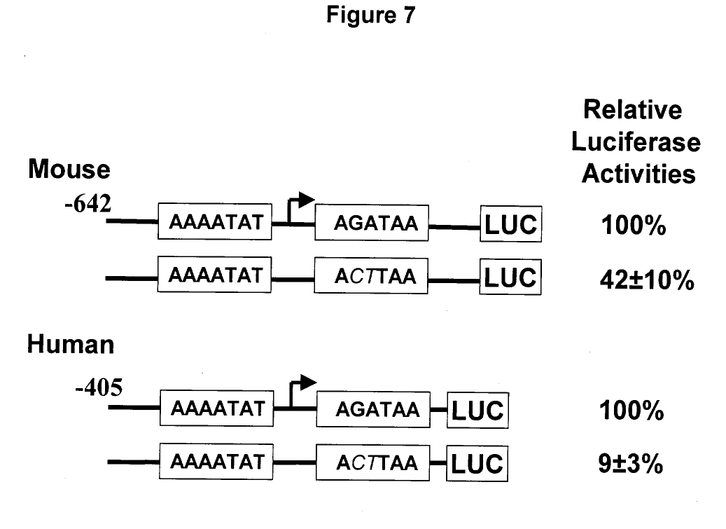

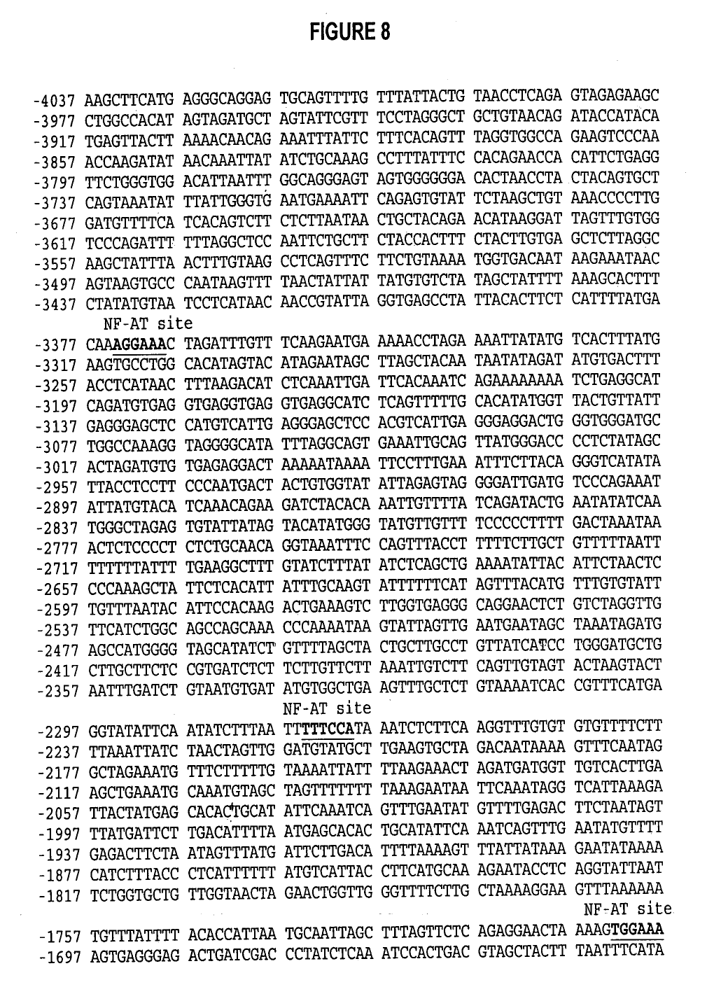

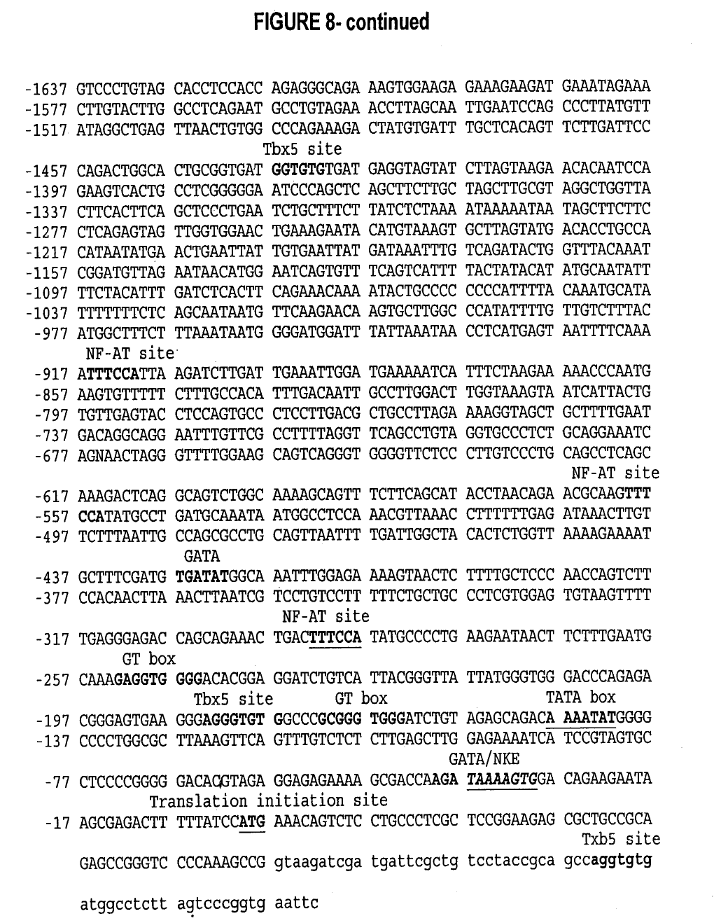

To determine whether the proximal GATA sequences indeed bind to GATA proteins, we prepared nuclear extracts from exponentially growing HL-5 cells as described (Schreiber E. et al., (1989) Nucleic Acids Res. 17:6419) and performed a competition electrophoretic mobility shift assay (EMSA) using a well-characterized consensus GATA oligonucleotide probe (Redondo, J. M. et al. (1990) Science 247(4947), 1225-9) and probes encompassing each of the proximal GATA sequences (FIG. 6A). The double-stranded oligonucleotide probes containing two consensus GATA sequences or mutated GATA sequences (GATA to CTTA) were purchased from Santa Cruz Biotechnology. The probes (see FIG. 6A) encompassing human or murine corin GATA (SEQ ID NOS: 11 and 13, respectively), or mutated human and murine corin GATA (GATA to CTTA) (SEQ ID NOS: 12 and 14, respectively), sequences were synthesized and HPLC-purified. The oligonucleotide probes were 5′-end-labeled with T4 polynucleotide kinase (Life Techologies) using [gamma-³²P ATP] (3000 Ci/mmol, Amersham Pharmacia Biotech). Gel mobility shift assays were performed as described previously (Pan, J. & McEver R. P. (1993) J. Biol. Chem. 268:22600-22608). As expected, the labeled consensus GATA probe (SEQ ID NO: 15) formed a sequence-specific DNA-protein complex when incubated with nuclear extracts of HL-5 cells (FIG. 6B). The formation of this complex was prevented by addition of a 100-fold excess of the unlabeled probe but not of an unrelated GAS element. The complex formation was dependent on the intact GATA sequence, because mutations in the GATA sequence abolished the formation of the complex. Furthermore, the complex was not detected in the presence of a 100-fold excess of the unlabeled probe containing either the human or murine proximal GATA sequences. In contrast, a 100-fold excess of the unlabeled probes encompassing the mutant proximal GATA sequences (SEQ ID NOS: 12 and 14) had minimal effect on the complex formation. These data indicate that the corin proximal GATA sequences and the consensus GATA probe bind to a common GATA protein(s).

To determine which GATA protein(s) was involved in the complex, we performed EMSA with the labeled consensus GATA probe in the presence of antibodies against members of the GATA family. Antibodies against mouse GATA-1 (SC-1234x), GATA-3 (SC-268x), GATA-4 (SC-12237x) and GATA-6 (SC-7244x) were from Santa Cruz Biotechnology (Santa Cruz, Calif.). As shown in FIG. 6C, an antibody against GATA-4 markedly inhibited the complex formation, whereas antibodies against GATA-1, -3 and -6 had little effect. To directly demonstrate the binding of GATA-4 to the proximal GATA sequence, we used the labeled human proximal GATA probe in the absence or presence of the same antibody against GATA4. As shown in FIG. 6D, the antibody against GATA-4 completely inhibited the formation of a DNA-protein complex with a similar mobility to that of the complex formed with the consensus GATA probe. These data indicate that GATA-4 bound to the proximal GATA sequences, suggesting that the binding of GATA-4 to the proximal GATA sequences may contribute to the gene expression of corin in cardiac myocytes.

To corroborate whether the proximal GATA elements are actually required for the promoter activity, we mutated the wild-type sequence AGATAA to ACTTAA in the human or murine constructs that promoted the highest promoter activity (FIG. 7). The mutant constructs, hCp405mutGATA and mCp646mutGATA, were constructed by an overlap PCR protocol (Ho S. N. et al., (1989) Gene (Amst) 77:51-59). Briefly, two separate PCR products, one for each half of the hybrid product, were generated with either an antisense or sense mutated GATA oligonucleotide and one outside primer. The two products were purified and mixed. A second PCR was then performed using the two outside primers. The PCR product was digested with Sac I and Hind III, and ligated into Sac I- and Hind III-digested pGL3-basic vector. All constructs were confirmed by restriction mapping and DNA sequencing. The mutations in the GATA element were the same as those made in the mutant GATA probes used in the EMSAs. When transfected into HL-5, the human and murine mutant constructs had 10% or 42% of promoter activities as compared to their respective wild type sequences. These results show that the proximal GATA elements are required for constitutive expression of the human or murine corin genes in cultured cardiomyocytes.

The preceding examples can be repeated with similar success by substituting the generically or specifically described reactants and/or operating conditions of this invention for those used in the preceding examples.

While the invention has been illustrated with respect to novel expression control regions for the human or mouse corin gene, it is apparent that variations and modifications of the invention can be made without departing from the spirit or scope of the invention.

What is claimed is:

1. An isolated polynucleotide comprising a mammalian corin expression control region, wherein the control region modulates the transcription of a heterologous polynucleotide to which it is operably linked.

2. The polynucleotide of claim 1, wherein the control region modulates transcription of a mammalian corin gene.

3. The polynucleotide of claim 1, wherein the transcription occurs in cardiac tissue.

4. The polynucleotide of claim 1, wherein the control region comprises a transcription regulation element, selected from the group consisting of GATA, Tbx-5, NKx2.5, or NF-AT binding sites.

5. The polynucleotide of claim 1, wherein the control region binds to transcription regulatory proteins, wherein the proteins are selected from the group consisting of GATA-4, Tbx-5, Nkx2.5, Krppel-like factor, or NF-AT transcription factor.

6. The polynucleotide of claim 1, wherein the mammal is a human.

7. The polynucleotide of claim 6, wherein the polynucleotide has the sequence set forth in SEQ ID NO: 4.

8. The polynucleotide of claim 6, wherein the polynucleotide has the sequence set forth in SEQ ID NO: 5.

9. The polynucleotide of claim 6, wherein the polynucleotide has the sequence set forth in SEQ ID NO: 6.

10. A fragment or variant of the polynucleotide of claim 1, wherein the fragment or variant is capable of transcribing the heterologous polynucleotide to which it is operably linked.

11. The fragment or variant of claim 10, wherein the fragment or variant has a sequence which is at least 70% identical to SEQ ID NO: 4.

12. The fragment or variant of claim 10, wherein the fragment or variant has a sequence which is at least 70% identical to SEQ ID NO: 5.

13. The fragment or variant of claim 10, wherein the fragment or variant has a sequence which is at least 70% identical to SEQ ID NO: 6.

14. A vector comprising the corin expression control region of claim 1.

15. A host cell comprising the vector of claim 14.

16. A method of producing a polypeptide comprising expressing from the host cell of claim 15 a polypeptide encoded by a heterologous polynucleotide operably linked to the corin expression control region.

17. A method of producing a polynucleotide comprising expressing from the host cell of claim 15 an antisense molecule encoded by a heterologous polynucleotide operably linked to the corin expression control region.

18. A pharmaceutical composition comprising the vector of claim 14 in a pharmaceutically acceptable carrier.

19. A pharmaceutical composition comprising the cell of claim 15 in a pharmaceutically acceptable carrier.

20. A method of identifying an agent which modulates the expression of a human corin gene in a cell, wherein the method comprises:
(a) producing a recombinant vector in which an isolated polynucleotide comprising a mammalian corin expression control region is operably linked to a reporter gene;
(b) transfecting the cell with the recombinant vector;
(c) treating the cell with the agent;
(d) measuring the level of expression of the reporter sequence in the treated cell; and
(e) comparing the level of expression of the reporter sequence in the presence of the agent to the level of expression in an transfected control cell which has not been treated

21. The method of claim 20, wherein the cell is a cardiac myocyte cell.

22. A method for modulating the expression of a gene in a human subject, the method comprising:
(a) producing a recombinant vector in which an isolated polynucleotide comprising a mammalian corin expression control region is operably linked to a heterologous polynucleotide;
(b) administering the vector in a therapeutically effective amount to the subject.

23. The method of claim 22, wherein the heterologous polynucleotide encodes a therapeutic protein such as corin, ANP, B-type natriuretic peptide, phospholamban, ACE, or negative dominant forms of these genes.

24. The method of claim 23, wherein the heterologous polynucleotide encodes corin.

25. The method of claim 22, wherein the heterologous polynucleotide encodes a therapeutic polynucleotide such as an antisense RNA molelcule or a catalytic RNA molecule.

26. The method of claim 22, wherein the control region is selected from the group consisting of SEQ ID NO: 4, SEQ ID NO: 5, or SEQ ID NO: 6.

27. A method for treating congestive heart failure, hypertension or myocardial infarction in a human subject, the method comprising administering a therapeutically effective amount of an isolated polynucleotide comprising a mammalian corin expression control region, operably linked to a gene selected from the group consisting of corin, ANP, B-type natriuretic peptide, phospholamban, ACE, or negative dominant forms of these genes, to the subject.

28. The method of claim 27, wherein the gene is corin.
